(19) United States
(12) Patent Application Publication
(10) Pub. No.: US 2026/0315475 A1
(43) Pub. Date: Oct. 15, 2026

(54) Method of Administering Belumosudil for Patent Subpopulations with Hepatic Impairment

(71) Applicant: Kadmon Corporation, LLC, Morristown, NJ (US)

(72) Inventor: Jasminder Sahi, Cambridge, MA (US)

(73) Assignee: Kadmon Corporation, LLC, Morristown, NJ (US)

(21) Appl. No.: 19/681,774

(22) Filed: May 19, 2026

Related U.S. Application Data
(63) Continuation of application No. PCT/US2023/080712, filed on Nov. 21, 2023.

(51) Int. Cl. A61K 31/517; A61P 37/06

(52) U.S. Cl. CPC A61K 31/517; A61P 37/06

(57) ABSTRACT

The present disclosure provides methods of administering belumosudil and a pharmaceutically acceptable salt thereof for the treatment of chronic graft-versus-host disease in patient populations with normal hepatic function and with hepatic impairment.

### TECHNICAL FIELD

The present disclosure relates to methods of administering belumosudil and a pharmaceutically acceptable salt thereof to treat chronic graft-versus-host disease in subjects with normal hepatic function and with hepatic impairment.

### BACKGROUND

Chronic graft-versus-host disease (cGVHD) is an immune-mediated inflammatory and fibrotic disorder. It is a potential, serious complication following solid organ transplant and allogeneic hematopoietic cell transplant (alloHCT). cGVHD affects up to 70% of all alloHCT recipients, with an incidence of 20%-50% in children. It is the leading cause of non-relapse mortality beyond 2 years after alloHCT. The estimated prevalence of cGVHD is 14,000 patients in the United States (as of 2016). (Bachier C R et al: Epidemiology and real-world treatment of chronic graft-versus-host disease post allogeneic hematopoietic cell transplantation: A US claims analysis. Presented at ASH 2019, Orlando, FL, Dec. 7-10, 2019) (“Bachier et al.”).

Patients with cGVHD have substantial impairment in quality of life (QOL) as assessed by the Lee Symptom Scale (LSS), which measures the effect of cGVHD on patients' functioning and well-being. It is reported that only one third of patients who have cGVHD and start systemic treatment will be alive, in remission and off immunosuppressive therapy by 5 years. (Lee S J et al: Success of immunosuppressive treatments in patients with chronic graft-versus-host disease. Biol Blood Marrow Transpl 24:555-562, 2018) (“Lee et al.”).

The pathophysiology of cGVHD can be separated into three phases: early inflammation because of tissue injury, a dysregulated adaptive immune system, and chronic inflammation and aberrant tissue repair with fibrosis.

First-line therapy for National Institutes of Health (NIH)-defined moderate to severe chronic graft-versus-host disease (cGVHD) is corticosteroids alone or in combination with sirolimus or a calcineurin inhibitor. However, up to 70% of patients require additional lines of therapy. (Bachier et al). Furthermore, the long-term use of corticosteroids is associated with significant side effects. (Lee et al).

Management of cGVHD continues to evolve with the advent of targeted therapies. cGVHD is characterized by an overproduction of proinflammatory cytokines IL-21 and IL-17, as well as overactivation of T follicular helper cells and B cells, which in turn leads to overproduction of antibodies.

In 2017, the US Food and Drug Administration approved ibrutinib, a Bruton's Tyr kinase inhibitor, for the treatment of adults with cGVHD after failure of one or more 1 systemic lines of therapy. In patients with cGVHD who were required to have either >25% body surface area erythematous rash or an NIH mouth score of >4, a study with ibrutinib reported an overall response rate (ORR) of 67% and a discontinuation rate because of treatment-emergent adverse events (TEAEs) of 43%. (Waller E K, et al: Ibrutinib for chronic graft-versus-host disease after failure of prior therapy: 1-Year update of a phase 1b/2 study. Biol Blood Marrow Transpl 25:2002-2007, 2019).

There remains an opportunity to study other treatment options for patients with cGVHD, including those who have failed ≥1 lines of therapy.

### SUMMARY

The present disclosure provides methods of administering belumosudil and a pharmaceutically acceptable salt thereof to treat chronic graft-versus-host disease in subjects with normal hepatic function and with hepatic impairment.

In one embodiment, the present disclosure provides for a method of treating chronic graft-versus-host disease (cGVHD) in a patient in need thereof, comprising the steps of verifying the patient's hepatic function and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is normal hepatic function or mild hepatic impairment (A on the Child-Pugh scale).

In one embodiment, the present disclosure provides for a method of treating chronic graft-versus-host disease (cGVHD) in a patient in need thereof, comprising the steps of verifying the patient's Child-Pugh score and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 0 to 6.

In another embodiment, the present disclosure provides for a method of treating chronic graft-versus-host disease (cGVHD) in a patient in need thereof, comprising the steps of verifying the patient's Child-Pugh score and not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 7 to 14 (B or C on the Child-Pugh scale).

In another embodiment, the present disclosure provides for a method of treating chronic graft-versus-host disease (cGVHD) in a patient in need thereof, comprising the steps of verifying the patient's Child-Pugh score and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is not 7 to 14 (B or C on the Child-Pugh scale).

In another embodiment, the present disclosure provides for a method of treating chronic graft-versus-host disease (cGVHD) in a patient in need thereof, comprising the steps of verifying the patient's hepatic function and not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is moderate or severe hepatic impairment (B or C on the Child-Pugh scale).

In another embodiment, the present disclosure provides for a method of treating chronic graft-versus-host disease (cGVHD) in a patient in need thereof, comprising the steps of verifying the patient's Child-Pugh score and not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 7 to 14 (B or C on the Child-Pugh scale).

In another embodiment, the present disclosure provides for a method of treating chronic graft-versus-host disease (cGVHD) in a patient in need thereof, comprising determining whether the patient has normal hepatic function or mild hepatic impairment, and when the patient is found to have normal hepatic function or mild hepatic impairment, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

In another embodiment, the present disclosure provides for a method of treating chronic graft-versus-host disease (cGVHD) in a patient in need thereof, comprising determining the patient's Child-Pugh score, and when the patient is found to have a Child-Pugh score of 5 or 6, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

In another embodiment, the present disclosure provides for a method of treating chronic graft-versus-host disease (cGVHD) in a patient in need thereof, comprising determining whether the patient has moderate or severe hepatic impairment, and when the patient is found not to have moderate or severe hepatic impairment, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

In another embodiment, the present disclosure provides for a method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient has normal hepatic function or mild hepatic impairment

In another embodiment, the present disclosure provides for a method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient does not have moderate or severe hepatic impairment

In another embodiment, the present disclosure provides for a method of treating chronic graft-versus-host disease (cGVHD) in a patient in need thereof, comprising administering to a patient in need thereof a therapeutically effective amount of Compound, wherein the patient does not have score of B or C on the Child-Pugh scale.

In another embodiment, the present disclosure provides for a method of treating chronic graft-versus-host disease (cGVHD) in a patient in need thereof, comprising the steps of measuring the patient's hepatic function a first time; administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is mild hepatic impairment (A on the Child-Pugh scale) and not administering Compound when the hepatic function of the patient is moderate or severe hepatic impairment (B or C on the Child-Pugh scale); measuring the patient's hepatic function a second time, wherein the second time is at least a month after the first time; and ceasing administration of the Compound when the hepatic function of the patient is moderate or severe hepatic impairment (B or C on the Child-Pugh scale), wherein measuring the hepatic function comprises measurement of at least one of the following: bilirubin, aspartate aminotransferase (AST), and alanine aminotransferase (ALT).

The present disclosure and further embodiments can be understood more fully by reference to the detailed description and examples, which are intended to exemplify non-limiting embodiments.

### BRIEF DESCRIPTION OF THE DRAWINGS

FIG. 1 is the arithmetic mean (+SD) plasma concentration-time profile of belumosudil (KD025) in a clinical phase I, single-dose, open-label, pharmacokinetic study in participants with normal hepatic function and participants with varying degrees of hepatic impairment, shown in linear scale.

FIG. 2 is the arithmetic mean (+SD) plasma concentration-time profile of belumosudil (KD025) in a clinical phase I, single-dose, open-label, pharmacokinetic study in participants with normal hepatic function and participants with varying degrees of hepatic impairment, shown in semi-logarithmic scale.

FIG. 3 is the arithmetic mean (+SD) plasma concentration-time profile of a belumosudil metabolite (KD025 m1) in a clinical phase I, single-dose, open-label, pharmacokinetic study in participants with normal hepatic function and participants with varying degrees of hepatic impairment, shown in linear scale.

FIG. 4 is the arithmetic mean (+SD) plasma concentration-time profile of a belumosudil metabolite (KD025 m1) in a clinical phase I, single-dose, open-label, pharmacokinetic study in participants with normal hepatic function and participants with varying degrees of hepatic impairment, shown in semi-logarithmic scale.

FIG. 5 is the arithmetic mean (+SD) plasma concentration-time profile of a belumosudil metabolite (KD025m2) in a clinical phase I, single-dose, open-label, pharmacokinetic study in participants with normal hepatic function and participants with varying degrees of hepatic impairment, shown in linear scale.

FIG. 6 is the arithmetic mean (+SD) plasma concentration-time profile of a belumosudil metabolite (KD025m2) in a clinical phase I, single-dose, open-label, pharmacokinetic study in participants with normal hepatic function and participants with varying degrees of hepatic impairment, shown in semi-logarithmic scale.

### DETAILED DESCRIPTION

### Overview

Belumosudil is an oral selective rho-associated coiled-coil-containing protein kinase-2 (ROCK2) inhibitor. ROCK2 inhibition acts on the dysregulated adaptive immune system and the fibrosis that occurs because of aberrant tissue repair. Belumosudil inhibits ROCK2 and ROCK1 with IC₅₀ values of approximately 100 nM and 3 μM, respectively.

Belumosudil down-regulates proinflammatory responses via regulation of STAT3/STAT5 phosphorylation and shifting Th17/Treg balance in ex-vivo or in vitro-human T cell assays. Belumosudil also inhibits aberrant pro-fibrotic signaling, in vitro. By controlling ROCK2 activity, belumosudil mediates signaling in immune cellular function and fibrotic pathways, thereby alleviating the effects caused by this debilitating disease, such as inflammation of multiple tissues and fibrotic changes that may involve several organs including the lungs, hepatobiliary system, musculoskeletal system, gastrointestinal (GI) tract, and skin. In vivo, belumosudil demonstrates activity in animal models of chronic GVHD.

The mesylate salt of belumosudil is marketed as REZUROCK™ in the United States and other countries for the treatment of patients with chronic GVHD (cGVHD), in some instances after failure of at least two prior lines of systemic therapy. The compound belumosudil has the chemical name: 2-{3-[4-(1H-indazol-5-ylamino)-2-quinazolinyl]phenoxy}-N-(propan-2-yl) acetamide. The compound belumosudil is also known as KD025. The active pharmaceutical ingredient of REZUROCK™ is belumosudil mesylate salt with the molecular formula C₂₇H₂₈N₆O₅S, a molecular weight of 548.62 g/mol, and having the chemical name 2-{3-[4-(1H-indazol-5-ylamino)-2-quinazolinyl]phenoxy}-N-(propan-2-yl) acetamide methanesulfonate (1:1).

The chemical structure of belumosudil mesylate is as follows:

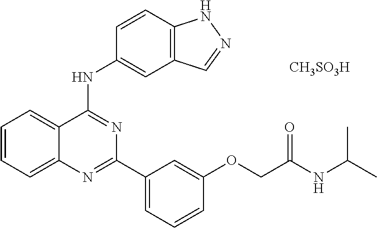

Belumosudil and processes for making the compound are described in the following US patents: U.S. Pat. Nos. 8,357,693, 9,815,820, 10,183,931, and 10,696,660.

The present disclosure provides methods of administering belumosudil and a pharmaceutically acceptable salt thereof to cGVHD patients with normal and impaired hepatic function.

### Definitions

“About” as used herein includes the exact amount modified by the term, about, as wells as an amount that would be expected to be within experimental error, such as for example, within 15%, 10%, or 5%. For example, “about 200 mg” means “200 mg” and also a range of mgs that is within experimental error, e.g., plus or minus 15%, 10%, or 5% of 200 mg. As used herein, the term “about” may be used to modify a range and also, a particular value.

“Administering” or “administered to” as used herein (for example, including use of this term with reference to cease administration, and/or resuming administration, of API, including Compound or belumosudil, to a subject), refers to the act of prescribing medicine(s) containing the API for the subject to take during treatment, the act of dispensing the medicine(s) to the subject, and/or the act of physically receiving or ingesting the medicine(s). Thus, the API (e.g., Compound or belumosudil), can be “administered” by a physician or other medical professional who writes prescriptions for medicine(s); and/or by a pharmacist who fills said prescriptions and/or dispenses the medicine(s) to the subject; and/or by the patient or subject who ingests the medicine and/or his or her partner or caretaker who provides the medicine to a subject, each of whom also may “cease” administration and/or “resume” administration of the API.

“API” means “active pharmaceutical ingredient.”

When the term “belumosudil” is used herein, it should be understood that, unless the context clearly indicates otherwise, the term may cover the compound belumosudil in any form as well as pharmaceutically acceptable salts thereof. The term “belumosudil” refers both to the compound belumosudil (for example, in the free base form, amorphous form, or crystalline form), to pharmaceutically acceptable salts of belumosudil, for example, the mesylate salt form as used in as REZUROCK,™ and to any form of belumosudil that may be used in a formulation or pharmaceutical composition for administering the compound to a patient.

“Ceasing” or “cessation” when used regarding administration of an API means that the API is no longer being administered to the patient on either a temporary or permanent basis. For example, if the patient experiences moderate or severe hepatic impairment (Child-Pugh B or C), administration of the API may be “ceased.” The cessation can be temporary, or administration can be permanently ceased or discontinued.

“Child-Pugh score,” “Child-Pugh class,” and “Child-Pugh scale” refer to a system for assessing the prognosis of chronic liver disease, including cirrhosis. The score is used to forecast the increasing severity of the liver disease and expected survival rate and to determine the required strength of treatment. Scores of 5 to 6 points correspond to mild hepatic impairment (Child-Pugh Class A); 7 to 9 points correspond to moderate hepatic impairment (Child-Pugh Class B); and 10 to 14 points correspond to severe hepatic impairment (Child-Pugh Class C). In one embodiment, 10 to 15 points correspond to severe hepatic impairment (Child-Pugh Class C).

In assessing the Child-Pugh score, the following criterion are used, with variable points for each criterion based on increasing severity: Encephalopathy: None=1 point, Grade 1 and 2=2 points, Grade 3 and 4=3 points; Ascites: None=1 point, slight=2 points, moderate=3 points; Bilirubin: under 2 mg/ml=1 point, 2 to 3 mg/ml=2 points, over 3 mg/ml=3 points; Albumin: greater than 3.5 mg/ml=1 point, 2.8 to 3.5 mg/ml=2 points, less than 2.8 mg/ml=3 points; Prothrombin Time* (sec prolonged): less than 4 sec=1 point, 4 to 6 sec=2 points, over 6 sec=3 points. *INR may be used as a substitute for PT, with INR under 1.7=1 point, INR 1.7 to 2.2=2 points, INR above 2.2=3 points.

“Clinical endpoint” or “study endpoint” refers to an event or outcome in a clinical trial that can be measured objectively to determine outcomes and potential beneficial effects of the drug or administration protocol as designed in the clinical trial. Examples of clinical endpoints include the following. Overall response rate (ORR) is the percentage of people in a study or treatment group who have a partial response (PR) or complete response (CR) to the treatment within a certain period of time. Failure-free survival (FFS) means the time from the first dose of belumosudil to a failure event, or the interval between the start of belumosudil and the addition of a new cGVHD therapy, relapse of the underlying disease, or nonrelapse mortality (NRM). Overall survival (OS) means the length of time from either the date of diagnosis or the start of treatment for a disease. Duration of response (DOR) means from the time of initial response (e.g., PR or CR) until documented progression from best response of cGVHD, time from initial response to start of additional systemic cGVHD therapy, or death. Time to next treatment (TTNT) means time to initiation of a subsequent systemic cGVHD therapy.

“Clinically recommended amount” or “clinically recommended dose” refers to the amount or dosage of API that has been recommended and/or approved for administration to a patient by those skilled in the field of medicinal chemistry to treat the disease state in question following clinical trials, for example, as described in Examples 1 and 2 hereof. In some embodiments, the clinically recommended dose of belumosudil mesylate is 200 mg a day, in some embodiments, 200 mg a day orally administered with food. In some embodiments, the clinically recommended dose of belumosudil mesylate is about 200 mg a day, in some embodiments, about 200 mg a day orally administered with food.

“CYP3A” refers to the CYP3A family of p-450 isoenzymes including CYP3A4.

“Immunosuppressive therapy” (IST) refers to therapy that is typically administered for at least six months after allo-HSCT to try to prevent GVHD. Examples of IST's include sirolimus, prednisone and calcineurin inhibitors such as tacrolimus and cyclosporine.

Lee Symptom Scale (LSS) summary score measures the effect on patients' functioning and well-being. The Lee Symptom Scale is a 30-item scale developed to measure the symptoms of cGVHD and is described in Lee S J, et al., Development and validation of a scale to measure symptoms of chronic graft-versus host disease. Biol Blood Marrow Transplant 2002; 8:444-452.

“Line of treatment” or “line of therapy” describes the sequence or order in which different therapies are given to a patient as the patient's disease progresses. Initial treatment (first-line therapy) may not work or may stop working after a period. After first-line therapy is discontinued, a second different treatment (second-line therapy) may be given. Subsequent lines of therapy may be given when a second-line therapy does not work or stops working. Some patients may be administered multiple lines of therapy over the course of a disease.

First-line therapy for National Institutes of Health (NIH)-defined moderate to severe chronic graft-versus-host disease (cGVHD) may be corticosteroids alone or in combination with sirolimus or a calcineurin inhibitor. (Carpenter P A, et al.: A phase II/III randomized, multicenter trial of prednisone/sirolimus versus prednisone/sirolimus/calcineurin inhibitor for the treatment of chronic graft-versus-host disease: BMT CTN 0801. Haematologica 103:1915-1924, 2018).

Examples of corticosteroid therapies for treatment of cGVHD include, but are not limited to, prednisone, prednisolone, methylprednisolone, and budesonide. Examples of prior systemic therapies for treating cGVHD include, but are not limited to, prednisone, tacrolimus, extracorporeal photopheresis (ECP), sirolimus, ibruitinib, ruxolitinib, mycophenolate mofetil (MMF), rituximab, methotrexate (MTX), cyclosporine, imatinib, ixazomib, and ofatumumab.

“Measuring” and “verifying” with reference to assessment of a subject's hepatic function means ascertaining, checking, establishing, confirming and/or assessing the status of the patient's hepatic function. The measuring or verifying can be performed via consultation, patient self-reporting, visual observation, physical examination, use of devices, laboratory testing, and/or any other means for detecting hepatic function, directly or indirectly. Any person who may be involved with administration of the API to a subject, as defined above, may also be involved with measuring and verifying hepatic function.

“Monitoring” with reference to assessment of a subject's hepatic function means observing, checking, and/or assessing the progression of the patient's hepatic function over at least two points in time; in some embodiments, over a period of time; in some embodiments, monthly; in some embodiments, at least monthly. The monitoring can be performed via consultation, patient self-reporting, visual observation, physical examination, use of devices, laboratory testing, and/or any other means for detecting hepatic function, directly or indirectly. Any person who may be involved with administration of the API to a subject, as defined above, may also be involved with monitoring hepatic function.

“Or” is used in the inclusive sense (equivalent to “and/or”) unless the context requires otherwise.

“Patient” or “subject” as used herein includes an animal or a human, in one embodiment, a human.

“Pharmaceutical composition” means a mixture of substances suitable for administering to an individual that includes a pharmaceutical agent. For example, a pharmaceutical composition may comprise a sterile aqueous solution or the API formulated in an oral dosage form such as a tablet or capsule.

“Pharmaceutically acceptable salt” means a physiologically and pharmaceutically acceptable salt of a compound provided herein. A “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

“Steroid-refractory” (SR) cGVHD is defined as cGVHD progression while on steroids or corticosteroids; in one embodiment, while on prednisone.

A “therapeutically effective amount” of an API means an amount which, when administered to a human for treating a disease (for example, cGVHD), is sufficient to effect treatment for the disease state being treated. As applied to cGVHD in a human, “treating” or “treatment” includes (1) reducing the risk of developing cGVHD and/or inhibiting cGVHD, i.e., arresting or reducing the development of cGVHD or its clinical symptoms; and (2) relieving cGVHD, i.e., causing regression, reversal, or amelioration of the cGVHD or reducing the number, frequency, duration or severity of its clinical symptoms.

The therapeutically effective amount of an API may vary depending upon the health and physical condition of the subject to be treated, the extent of disease progression, the assessment of the medical situation, and other relevant factors. It is expected that the therapeutically effective amount may fall within a range that can be determined through trial and through reference to clinical trial data and results, for example, as described in Examples 1 and 2 hereof and in scientific literature.

### EXEMPLARY EMBODIMENTS

In one embodiment, the present disclosure provides for a method of treating chronic graft-versus-host disease (cGVHD) in a patient in need thereof, comprising the steps of verifying the patient's hepatic function and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is normal hepatic function or mild hepatic impairment (A on the Child-Pugh scale).

In one embodiment, the patient's hepatic function is normal.

In one embodiment, the patient's hepatic function is mild hepatic impairment.

In one embodiment, the present disclosure provides for a method further comprising the step of ceasing the administration of the Compound to the patient when the patient's hepatic function indicates an increase in hepatic impairment.

In one embodiment, the patient's hepatic function changes from normal hepatic function to moderate or severe hepatic mild impairment (B or C on the Child-Pugh scale).

In one embodiment, the patient's hepatic function changes from mild hepatic impairment (A on the Child-Pugh scale) to moderate or severe hepatic impairment (B or C on the Child-Pugh scale).

In one embodiment, the normal hepatic function is defined as the patient scoring 0 to 4 points on the Child-Pugh scale, wherein mild hepatic impairment is defined as the patient scoring 5 or 6 points on the Child-Pugh scale, wherein moderate hepatic impairment is defined as the patient scoring 7 to 9 points on the Child-Pugh scale, and wherein severe hepatic impairment is defined as the patient scoring 10 to 14 points on the Child-Pugh scale.

In one embodiment, the normal hepatic function is defined as the patient scoring 0 to 4 points on the Child-Pugh scale, wherein mild hepatic impairment is defined as the patient scoring 5 or 6 points on the Child-Pugh scale, wherein moderate hepatic impairment is defined as the patient scoring 7 to 9 points on the Child-Pugh scale, and wherein severe hepatic impairment is defined as the patient scoring 10 to 15 points on the Child-Pugh scale.

In one embodiment, the present disclosure provides for a method of treating chronic graft-versus-host disease (cGVHD) in a patient in need thereof, comprising the steps of verifying the patient's Child-Pugh score and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 5 or 6.

In one embodiment, the present disclosure provides for a method further comprising the step of ceasing the administration of the Compound to the patient when the patient's Child-Pugh score increases.

In one embodiment, the present disclosure provides for a method further comprising the step of ceasing the administration of the Compound to the patient when the patient's Child-Pugh score increases to 7 or greater.

In one embodiment, the present disclosure provides for a method of treating chronic graft-versus-host disease (cGVHD) in a patient in need thereof, comprising the steps of verifying the patient's Child-Pugh score and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 0 to 4.

In one embodiment, the present disclosure provides for a method further comprising the step of ceasing the administration of the Compound to the patient when the patient's Child-Pugh score increases to 7 or greater.

In one embodiment, the present disclosure provides for a method of treating chronic graft-versus-host disease (cGVHD) in a patient in need thereof, comprising the steps of verifying the patient's hepatic function and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is not moderate or severe hepatic impairment (B or C on the Child-Pugh scale).

In one embodiment, the present disclosure provides for a method of treating chronic graft-versus-host disease (cGVHD) in a patient in need thereof, comprising the steps of verifying the patient's Child-Pugh score and administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is not 7 to 14 (B or C on the Child-Pugh scale).

In one embodiment, the present disclosure provides for a method of treating chronic graft-versus-host disease (cGVHD) in a patient in need thereof, comprising the steps of verifying the patient's hepatic function and not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is moderate or severe hepatic impairment (B or C on the Child-Pugh scale).

In one embodiment, the present disclosure provides for a method of treating chronic graft-versus-host disease (cGVHD) in a patient in need thereof, comprising the steps of verifying the patient's Child-Pugh score and not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 7 to 14 (B or C on the Child-Pugh scale).

In one embodiment, the present disclosure provides for a method of treating chronic graft-versus-host disease (cGVHD) in a patient in need thereof, comprising the steps of verifying the patient's Child-Pugh score and not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 7 to 15 (B or C on the Child-Pugh scale).

In one embodiment, the present disclosure provides for a method of treating chronic graft-versus-host disease (cGVHD) in a patient in need thereof, comprising determining whether the patient has normal hepatic function or mild hepatic impairment, and when the patient is found to have normal hepatic function or mild hepatic impairment, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

In one embodiment, the administration of Compound to the patient is ceased when the patient's hepatic function indicates an increase in hepatic impairment.

In one embodiment, the patient's hepatic function changes from normal hepatic function or mild hepatic impairment to moderate or severe hepatic impairment.

In one embodiment, the present disclosure provides for a method of treating chronic graft-versus-host disease (cGVHD) in a patient in need thereof, comprising determining the patient's Child-Pugh score, and when the patient is found to have a Child-Pugh score of 5 or 6, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

In one embodiment, the administration of Compound to the patient is ceased when the patient's Child-Pugh score increases to 7 or greater.

In one embodiment, the present disclosure provides for a method of treating chronic graft-versus-host disease (cGVHD) in a patient in need thereof, comprising determining whether the patient has moderate or severe hepatic impairment, and when the patient is found not to have moderate or severe hepatic impairment, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

In one embodiment, the present disclosure provides for a method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient has normal hepatic function or mild hepatic impairment.

In one embodiment, the administration of Compound to the patient is ceased when the patient's hepatic function indicates an increase in hepatic impairment.

In one embodiment, the patient's hepatic function changes from normal hepatic function or mild hepatic impairment to moderate or severe hepatic impairment.

In one embodiment, the present disclosure provides for a method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient does not have moderate or severe hepatic impairment.

In one embodiment, the present disclosure provides for a method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient has a score of A on the Child Pugh scale.

In one embodiment, the administration of Compound to the patient is ceased when the patient's hepatic function indicates an increase in hepatic impairment.

In one embodiment, the administration of Compound to the patient is ceased when the patient's hepatic function changes from a score of A to a score of B or C on the Child-Pugh scale.

In one embodiment, the present disclosure provides for a method of treating chronic graft-versus-host disease (cGVHD) in a patient in need thereof, comprising administering to a patient in need thereof a therapeutically effective amount of Compound, wherein the patient does not have score of B or C on the Child-Pugh scale.

In one embodiment, the present disclosure provides for a method of treating chronic graft-versus-host disease (cGVHD) in a patient in need thereof, comprising the steps of measuring the patient's hepatic function a first time; administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is mild hepatic impairment (A on the Child-Pugh scale) and not administering Compound when the hepatic function of the patient is moderate or severe hepatic impairment (B or C on the Child-Pugh scale); measuring the patient's hepatic function a second time, wherein the second time is at least a month after the first time; and ceasing administration of the Compound when the hepatic function of the patient is moderate or severe hepatic impairment (B or C on the Child-Pugh scale), wherein measuring the hepatic function comprises measurement of at least one of the following: bilirubin, aspartate aminotransferase (AST), and alanine aminotransferase (ALT).

In one embodiment, the second time is about a month after the first time.

In one embodiment, the second time is a month after the first time.

In one embodiment, measuring hepatic function comprises measurement of bilirubin.

In one embodiment, measuring hepatic function comprises measurement of total bilirubin.

In one embodiment, the patient does not have liver cGVHD.

In one embodiment, the present disclosure provides for a method of treating chronic graft-versus-host disease (cGVHD) in a patient in need thereof, wherein the cGVHD does not comprise liver cGVHD.

In one embodiment, the present disclosure provides for a method of treating chronic graft-versus-host disease (cGVHD) in a patient in need thereof, wherein the patient is without liver cGVHD.

In one embodiment, the present disclosure provides for a method of treating chronic graft-versus-host disease (cGVHD) in a patient in need thereof, wherein the patient is monitored for hepatic function at least monthly.

In one embodiment, the present disclosure provides for a method of treating chronic graft-versus-host disease (cGVHD) in a patient in need thereof, wherein the patient is monitored for bilirubin, aspartate aminotransferase (AST), and alanine aminotransferase (ALT).

In one embodiment, the present disclosure provides for a method of treating chronic graft-versus-host disease (cGVHD) in a patient in need thereof, wherein the patient is monitored for bilirubin and/or aspartate aminotransferase (AST) and/or alanine aminotransferase (ALT) at least monthly.

In one embodiment, the present disclosure provides for a method of treating chronic graft-versus-host disease (cGVHD) in a patient in need thereof, wherein the patient is monitored for total bilirubin and/or aspartate aminotransferase (AST) and/or alanine aminotransferase (ALT) at least monthly.

In one embodiment, the present disclosure provides for a method of treating chronic graft-versus-host disease (cGVHD) in a patient in need thereof, wherein the patient is monitored for bilirubin, aspartate aminotransferase (AST), and alanine aminotransferase (ALT) at least monthly.

In one embodiment, the present disclosure provides for a method of treating chronic graft-versus-host disease (cGVHD) in a patient in need thereof, wherein the patient is monitored for total bilirubin, aspartate aminotransferase (AST), and alanine aminotransferase (ALT) at least monthly.

In one embodiment, the present disclosure provides for a method of treating chronic graft-versus-host disease (cGVHD) in a patient in need thereof, wherein the Compound comprises belumosudil mesylate salt.

In one embodiment, the present disclosure provides for a method of treating chronic graft-versus-host disease (cGVHD) in a patient in need thereof, wherein the patient has failed at least two prior lines of systemic therapy for the cGVHD.

In one embodiment, the present disclosure provides for a method of treating chronic graft-versus-host disease (cGVHD) in a patient in need thereof, wherein the Compound is administered to the patient at a dose of 200 mg once daily.

In one embodiment, the present disclosure provides for a method of treating chronic graft-versus-host disease (cGVHD) in a patient in need thereof, wherein the Compound is administered to the patient at a dose of 200 mg twice daily.

In one embodiment, the present disclosure provides for a method of treating chronic graft-versus-host disease (cGVHD) in a patient in need thereof, wherein the Compound is administered to the patient at a dose of about 200 mg once daily.

In one embodiment, the present disclosure provides for a method of treating chronic graft-versus-host disease (cGVHD) in a patient in need thereof, wherein the Compound is administered to the patient at a dose of about 200 mg twice daily.

In one embodiment, the Compound is administered daily with food.

### Overview

The following abbreviations may be helpful in considering the Examples and description herein.

### Abbreviations

| | |
|---|---|
| AE | Adverse events |
| alloHCT | allogeneic hematopoietic cell |
| | transplantation |
| AUC | Area under the concentration time |
| | curve |
| BLQ | Below the limit of quantification |
| BM | Bone marrow |
| BMI | Body mass index |
| cGVHD | Chronic graft versus host disease |
| CI | Confidence interval |
| CR | Complete response |
| CV | Coefficient of variation |
| CYP | Cytochrome P450 |
| DDI | Drug-drug interaction |
| DOR | Duration of response |
| ECG | Electrocardiogram |
| FFS | Failure-free survival |
| GLSM | Geometric least squares mean |
| HLA | human leukocyte antigen |
| IMP | Investigational medicinal product |
| IST | Immunosuppressive therapy |
| LSS | Lee Symptom Scale |
| MR_{AUC} | Metabolite/parent ratio based on |
| | AUC |
| ORR | Overall response rate |
| OS | Overall survival |
| PBSC | peripheral blood stem cells |
| PK | Pharmacokinetic |
| PR | Partial response |
| QOL | Quality of life |
| SAE | Serious adverse event |
| SD | Standard deviation |
| SR | Steroid refractory |
| TEAEs | Treatment-emergent adverse events |
| TTNT | Time to next treatment |

### EXAMPLES

### Example 1: a Clinical Phase I, Single-Dose, Open-Label, Pharmacokinetic Study of KD025 in Participants with Normal Hepatic Function and Participants with Varying Degrees of Hepatic Impairment

### Objective

The primary objective was to assess the pharmacokinetic (PK) of belumosudil following a single oral dose of belumosudil in participants with mild, moderate, or severe hepatic impairment compared to healthy participants with normal hepatic function.

The secondary objectives were: to evaluate the safety and tolerability of a single oral dose of belumosudil in participants with mild, moderate, or severe hepatic impairment, and in healthy participants with normal hepatic function; to assess the PK of the belumosudil metabolites following a single oral dose of belumosudil in participants with mild, moderate, or severe hepatic impairment compared to healthy participants with normal hepatic function.

### Methodology

This was a Phase 1, open-label, non-randomized, parallel-group study to determine the effect of hepatic impairment on the PK, safety, and tolerability of a single oral dose of belumosudil compared to matched healthy participants with normal hepatic function.

Approximately 38 participants were to be enrolled into the following groups based on their hepatic function, as determined according to the Child-Pugh system.
Group 1 (n=approximately 16): matched healthy participants with normal hepatic function
Group 2 (n=8): participants with mild hepatic impairment (Child-Pugh Class A [score of 5 or 6])
Group 3 (n=8): participants with moderate hepatic impairment (Child-Pugh Class B [score of 7 to 9])
Group 4 (n=6): participants with severe hepatic impairment (Child-Pugh Class C [score of 10 to 14]).

Healthy participants with normal hepatic function (Group 1) were demographically matched by age (±10 years), sex, and body mass index (BMI; +20%) to participants with hepatic impairment (Groups 2, 3, and 4). Participants with normal hepatic function could be matched to multiple participants across the 3 hepatic impairment groups (mild, moderate, and severe), but could not be matched to more than 1 participant within the same impairment group.

Potential participants were screened to assess their eligibility to enter the study within 28 days prior to the dose administration. Participants were admitted into the clinical research unit (CRU) on Day −1 and were confined to the CRU until discharge on Day 3. Participants returned to the CRU for a follow-up visit on Day 10 (approximately 9 days after the belumosudil dose).

On the morning of Day 1, a single oral dose of 200 mg belumosudil was administered with 240 mL of water, within 5 minutes of completing a standardized breakfast. Pharmacokinetic blood samples were obtained from predose until 48 hours postdose (Day 3).

### Number of Participants

It was planned to study approximately 38 participants: 8 participants in each of the mild and moderate hepatic impairment groups, 6 participants in the severe hepatic impairment group, and approximately 16 matched participants with normal hepatic function.

A total of 36 participants were enrolled in the study in accordance with the protocol and protocol amendments: 8 participants in each of the mild and moderate hepatic impairment groups, 6 participants in the severe hepatic impairment group, and 14 matched participants with normal hepatic function. Data for all participants were included in the PK and safety analyses.

### Diagnosis and Main Criteria for Inclusion

Participants of any race, aged between 18 and 75 years, inclusive, and with a BMI between 18.0 and 38.0 kg/m², inclusive.

The matched control participants with normal hepatic functions were to be in good health, as assessed by the investigator (or designee), and the participants with hepatic impairment were to have documented chronic stable liver disease and may have had medical findings consistent with their hepatic dysfunction.

### Study Products

A single dose of 200 mg belumosudil was administered orally as a tablet following a standard breakfast.

### Duration of Treatment

Single doses were administered in each group.

### Criteria for Evaluation

The primary endpoints, PK endpoints of belumosudil derived from the concentration time profile following single-dose oral administration, were area under the concentration time curve (AUC) from time 0 to infinity (AUC_{0-∞}), AUC from time 0 to the last measurable concentration (AUC₀₋ₜ), maximum observed concentration (Cₘₐₓ), time of Cₘₐₓ (tₘₐₓ), terminal elimination half-life (t_{1/2}), time of last measurable concentration (tₗₐₛₜ), apparent total clearance (CL/F), and apparent volume of distribution during the terminal elimination phase (V_{Z}/F).

The secondary/safety endpoints were: incidence and severity of adverse events (AEs), clinical laboratory parameters (hematology, clinical chemistry, coagulation, and urinalysis), 12-lead electrocardiogram (ECG) parameters, vital signs parameters, and physical examinations.

The PK outcome endpoints of the belumosudil metabolites derived from the concentration-time profile following single-dose oral administration were as follows and were determined whenever possible: AUC_{0-∞}, AUC₀₋ₜ, Cₘₐₓ, tₘₐₓ, t_{1/2}, tₗₐₛₜ, and metabolite to parent ratios, adjusted for molecular weight.

### Statistical Methods

All PK concentrations and parameters were listed.

Summary tables, mean (+standard deviation [SD]) figures, overlaying individual figures, and individual figures by hepatic function and time postdose were provided for plasma PK concentrations. Summary tables by hepatic function were provided for all PK parameters, with the exception of terminal phase regression-related diagnostic parameters (λz, λz upper, λ z lower, λz number of points, R2-adj, and % AUCextrap), which were listed only.

A statistical analysis was conducted to evaluate the PK of belumosudil and metabolites following a single dose in participants with mild, moderate, or severe hepatic impairment (test hepatic functions) compared to their matched healthy participants with normal hepatic function (reference hepatic function) for primary PK parameters (AUC₀₋ₜ, AUC_{0-∞}, and Cₘₐₓ). All other PK parameters were regarded as secondary and were not participant to inferential statistical analysis.

All safety data for the safety population were listed.

Treatment-emergent AEs (TEAEs) were summarized by severity and relationship to the investigational medicinal product (IMP). The frequency of TEAEs was summarized by Medical Dictionary for Regulatory Activities system organ class and preferred term, and hepatic function. Summary and frequency TEAE tables were presented for all causalities and for those considered related to the IMP.

Serum biochemistry, hematology, and coagulation data were summarized by hepatic function. In addition, all serum biochemistry, hematology, and urinalysis data outside the clinical reference ranges were listed by parameter and treatment. Vital signs and ECG data were summarized by hepatic function, together with changes from baseline.

### Summary Results

### Population Characteristics

A total of 36 participants were enrolled in the study in accordance with the protocol and protocol amendments: 8 participants in each of the mild and moderate hepatic impairment groups, 6 participants in the severe hepatic impairment group, and 14 matched participants with normal hepatic function. Male (72.2%) and female (27.8%) participants aged 36 to 70, inclusive, with a BMI between 22.6 and 38.0 kg/m², inclusive, were enrolled in the study.

### Pharmacokinetic Results

The following PK analysis considerations were noted:

One participant (moderate hepatic impairment) did not complete the 48-hour postdose sample due to early discharge following an SAE. The PK was not affected as concentrations of all analytes were below the limit of quantification (BLQ) by 36 hours postdose.

One participant (mild hepatic impairment) had an embedded BLQ at 8 hours postdose for metabolite KD025m1. This concentration was excluded from descriptive statistics and PK analysis.

Two participants (normal hepatic function and moderate hepatic impairment) had full BLQ profiles for KD025m1; therefore, no PK parameters were calculable.

Seven participants had fewer than 3 consecutive quantifiable concentrations in their profiles; only Cₘₐₓ, t_{lag}, tₘₐₓ, and tₗₐₛₜ were reportable for: 4 participants with normal hepatic function and 2 participants with mild impairment for KD025m1; 1 participant with moderate impairment for KD025m2.

For multiple participants, a terminal phase regression could not be determined due to poor fit (r2 adj. <0.7); regression-based parameters (AUC_{0-∞}, % AUCₑₓₜᵣₐₚ, t_{1/2}, CL/F, and V_{z}/F) were not calculable for: 1 participant with severe impairment and 1 participant with normal hepatic function for KD025; 1 participant with mild impairment, 2 participants with moderate impairment, and 1 participant with severe impairment for KD025m1; 1 participant with severe impairment for KD025m2.

Terminal phase regression-based parameters for the following participants and analytes were listed but flagged for exclusion from statistics because % AUCₑₓₜᵣₐₚ>30%: 1 participant with severe impairment for KD025; 2 participants with normal hepatic function, 1 participant with mild impairment, 1 participant with moderate impairment for KD025m1; 3 participants with severe impairment for KD025m2.

For several participants, the half-life was calculated over a span of time less than twice the resultant half-life. These half-life values are flagged and footnoted but included in statistics.

Thirteen participants (2 with mild impairment, 5 with moderate impairment, and all 6 with severe impairment) were taking rifaximin and are flagged and footnoted.

Rifaximin is a non-absorbable rifampicin analog. Rifampicin is a strong CYP inducer and has been shown to decrease exposure of belumosudil by approximately 60% to 70% (KD025-107). Per label, rifaximin is not shown to have drug-drug interaction (DDI) effects in healthy volunteers, consistent with a non-absorbable compound, and DDI effects have not been reported in participants with hepatic impairment. However, in vitro, rifaximin has been shown to be a CYP3A4 inducer at high concentrations, which may result in a clinical DDI due to higher rifaximin exposure in participants with hepatic impairment. The potential for a clinical DDI effect has not been studied in participants with hepatic impairment. Rifaximin plasma concentration increases with Child-Pugh class, so participants with a greater degree of hepatic impairment may have higher rifaximin plasma levels than participants with mild hepatic impairment or healthy volunteers. If rifaximin levels were higher in moderately and severely impaired participants, higher levels of CYP induction could be expected, thus belumosudil exposure may be reduced. Rifaximin is a commonly used medication in participants with hepatic impairment; participants in this study were not in violation of the inclusion or exclusion criteria by taking this medication.

One participant with moderate impairment showed a greatly reduced concentration profile and exposure, which may be consistent with rifaximin dosing. This participant's belumosudil and metabolite concentrations and PK parameters are also flagged for exclusion from statistics and are participant to sensitivity analysis due to atypical concentration profiles.

Two participants with severe impairment show greatly reduced concentration profiles and exposures, which may be consistent with rifaximin dosing. These participants' belumosudil and metabolite concentrations and PK parameters are also flagged for exclusion from statistics and are participant to sensitivity analysis.

### Pharmacokinetic Analysis of Belumosudil

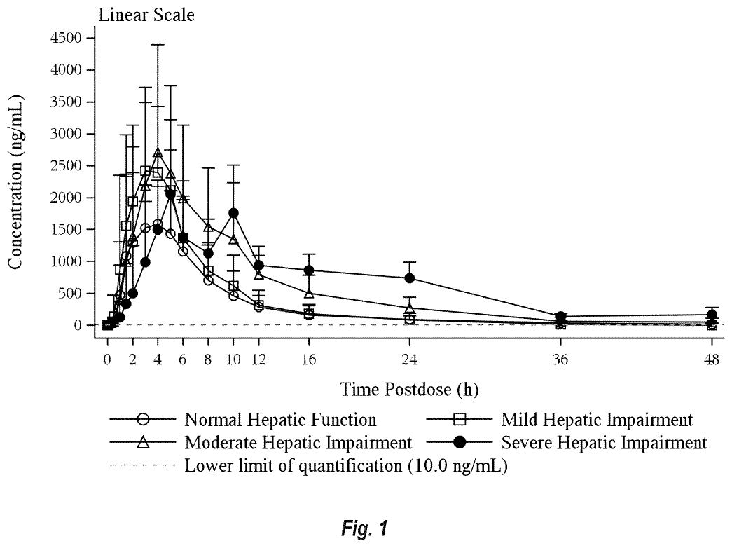

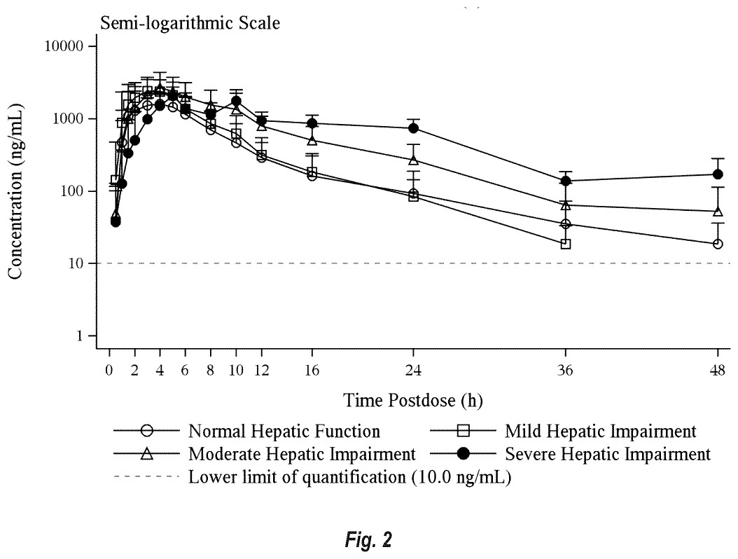

Arithmetic mean (+SD) plasma concentration-time profiles of belumosudil are presented in FIGS. 1 and 2. The summary and statistical analysis of plasma PK parameters of belumosudil are presented in Table 1.

TABLE 1
Summary of the Plasma Pharmacokinetic Parameters of Belumosudil

| | | | Moderate | |
|---|---|---|---|---|
| | Normal Hepatic | Mild Hepatic | Hepatic | Severe Hepatic |
| | Function | Impairment | Impairment | Impairment |
| Parameter | (N = 14) | (N = 8) | (N = 8) | (N = 6) |
| AUC_{(0-∞)} | 12800 (52.9) [13] | 16000 | 23700 (61.2) [7] | 30100 |
| (h*ng/mL) | | (45.3) [8] | | (14.7) [3] |
| AUC₍₀₋₂₄₎ | 11300 (49.5) [14] | 15400 | 21300 (57.7) [7] | 23400 |
| (h*ng/mL) | | (44.6) [8] | | (20.3) [4] |
| AUC₀₋ₜ | 12000 (53.1) [14] | 15800 | 23100 (60.9) [7] | 29600 |
| (h*ng/mL) | | (45.4) [8] | | (17.6) [4] |
| Cₘₐₓ | 2190 (36.4) [14] | 2590 (30.7) | 2490 (69.8) [7] | 2170 (33.6) |
| (ng/mL) | | [8] | | [4] |
| tₘₐₓ (h) | 3.50 | 2.98 | 4.00 | 5.02 |
| | (1.50-6.00) [14] | (1.92-4.00) | (3.00-8.00) [7] | (5.00-10.0) |
| | | [8] | | [4] |
| tₗₐₛₜ (h) | 48.0 | 36.0 | 48.0 | 48.0 |
| | (16.0-48.0) [14] | (16.0-48.0) | (23.8-48.0) [7] | (48.0-48.0) |
| | | [8] | | [4] |
| t_{1/2} (h) | 8.58 (48.8) [13] | 5.86 (45.2) | 6.30 (44.4) [7] | 10.00 (40.7) |
| | | [8] | | [3] |
| CL/F | 15.6 (52.9) [13] | 12.5 (45.3) | 8.42 (61.2) [7] | 6.63 (14.7) |
| (L/h) | | [8] | | [3] |
| V_{z}/F (L) | 193 (57.9) [13] | 105 (63.4) [8] | 76.6 (49.5) [7] | 95.7 (48.4) |
| | | | | [3] |
| t_{lag} (h) | NC (NC) [14] | NC (NC) [8] | NC (NC) [7] | NC (NC) [4] |

Matrix: Plasma;
Analyte: KD025
AUC_{0-∞} = area under the concentration-time curve from time 0 to infinity;
AUC₀₋ₜ = area under the concentration-time curve from time 0 to the time of the last quantifiable concentration;
CL/F = apparent total clearance;
Cₘₐₓ = maximum observed concentration;
CV = coefficient of variation (%);
n = number of participants with valid observations;
NC = not calculated;
t_{1/2} = apparent terminal elimination half-life;
tₗₐₛₜ = time of the last quantifiable concentration;
tₘₐₓ = time of the maximum observed concentration;
V_{z}/F = apparent volume of distribution during the terminal phase;
(%)AUCextrap = percentage of area under the concentration-time curve due to extrapolation
from the last quantifiable concentration to infinity
Geometric mean (CV) [n] statistics presented;
for tₘₐₓ, and tₗₐₛₜ, median (min-max) [n] statistics presented.

Following administration of a single oral dose of 200 mg belumosudil, the plasma concentration-time profiles of belumosudil in participants with normal hepatic function, and with mild, moderate, and severe hepatic impairment were characterized by steady absorption and bi-phasic disposition, with respective median tₘₐₓ values of 3.50, 2.98, 4.00, and 5.02 hours and geometric mean t_{1/2} values of 8.58, 5.86, 6.30, and 10.00 hours. The severe hepatic impairment group had delayed median tₘₐₓ and longer geometric mean t_{1/2} compared to other hepatic function groups.

Geometric mean CL/F values for belumosudil decreased as the severity of hepatic impairment increased, with values of 15.6, 12.5, 8.42, and 6.63 L/h for participants with normal hepatic function, and with mild, moderate, and severe hepatic impairment, respectively.

Geometric mean V_{z}/F values were generally lower for participants with hepatic impairment (76.6 to 105 L) than for participants with normal hepatic function (193 L).

Geometric mean PK exposure of belumosudil was higher in participants with hepatic impairment compared to participants with normal hepatic function. However, statistical analysis showed that the effect of mild and moderate impairment on belumosudil PK exposure was not statistically significant. The ratios of the geometric least squares mean (GLSM; 90% CI) for AUC_{0-∞}, AUC₀₋ₜ, and Cₘₐₓ were 1.36 (0.831, 2.21), 1.48 (0.960, 2.28), and 1.20 (0.907, 1.58), respectively, for the mild hepatic impairment group, and 1.51 (0.975, 2.33), 1.50 (0.973, 2.31), and 0.944 (0.600, 1.48), respectively, for the moderate hepatic impairment group, with 90% CIs spanning unity. However, severe hepatic impairment significantly increased the AUC_{0-∞} and AUC₀₋ₜ of belumosudil but had no apparent effect on its Cₘₐₓ, with the ratios of the GLSMs (90% CI) of 4.21 (2.20, 8.06), 3.23 (1.53, 6.81), and 1.32 (0.896, 1.96), respectively.

As assessed from the within-matched participants CV %, the variability was generally high for all comparisons for AUC_{0-∞}, AUC₀₋ₜ, and Cₘₐₓ, with values ranging from 23.5% to 54.9%.

### Pharmacokinetic Analysis of Belumosudil Metabolites

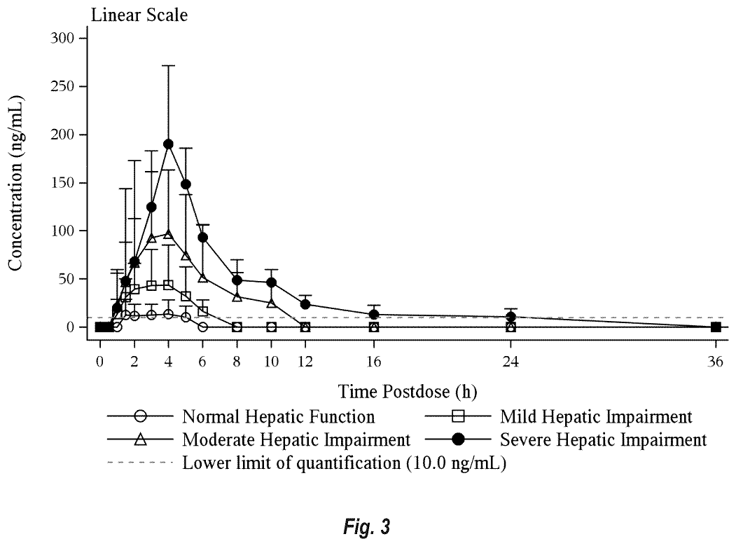

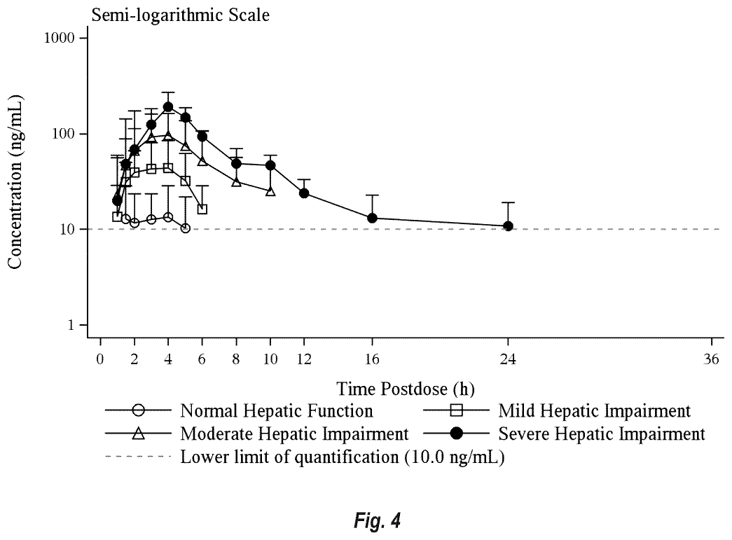

Arithmetic mean (+SD) plasma concentration-time profiles of KD025m1 are presented in FIGS. 3 and 4. The summary and statistical analysis of plasma PK parameters of KD025m1 are presented in Table 2, respectively.

TABLE 2
Summary of the Plasma Pharmacokinetic Parameters of KD025 ml

| | | | Moderate | |
|---|---|---|---|---|
| | Normal Hepatic | Mild Hepatic | Hepatic | Severe Hepatic |
| | Function | Impairment | Impairment | Impairment |
| Parameter | (N = 14) | (N = 8) | (N = 8) | (N = 6) |
| AUC_{(0-∞)} | NC (NC) [2] | 304 (21.0) [4] | 681 (66.4) [4] | 1180 (42.8) [3] |
| (h*ng/mL) | | | | |
| AUC₍₀₋₂₄₎ | 138 (36.0) [4] | 315 (40.5) [6] | 464 (105.3) [7] | 1030 (30.6) [4] |
| (h*ng/mL) | | | | |
| AUC₀₋ₜ | 71.6 (58.7) [9] | 242 (54.8) [6] | 382 (141.1) [7] | 1010 (32.7) [4] |
| (h*ng/mL) | | | | |
| Cₘₐₓ | 24.1 (50.4) [13] | 43.9 (100.9) [8] | 90.2 (78.2) [7] | 180 (48.0) [4] |
| (ng/mL) | | | | |
| tₘₐₓ (h) | 3.00 | 2.46 | 3.95 | 4.52 |
| | (1.50-6.00) [13] | (1.48-4.00) [8] | (1.50-5.00) [7] | (4.00-6.00) [4] |
| tₗₐₛₜ (h) | 5.00 | 6.00 | 10.0 | 24.0 |
| | (1.95-10.0) [13] | (1.48-10.0) [8] | (5.00-24.0) [7] | (12.0-24.1) [4] |
| t_{1/2} (h) | 2.29 (18.8) [4] | 2.46 (91.5) [5] | 3.06 (33.4) [5] | 5.36 (77.7) [3] |
| MR_{AUC} | 0.00696 (24.1) | 0.0147 (70.3) [6] | 0.0182 (91.6) [7] | 0.0377 (26.1) [4] |
| | [9] | | | |
| t_{lag} (h) | NC (NC) [13] | NC (NC) [8] | NC (NC) [7] | NC (NC) [4] |

Matrix: Plasma;
Analyte: KD025 ml
AUC_{0-∞} = area under the concentration-time curve from time 0 to infinity;
AUC₀₋ₜ = area under the concentration-time curve from time 0 to the time of the last quantifiable concentration;
CL/F = apparent total clearance;
Cₘₐₓ = maximum observed concentration;
CV = coefficient of variation (%);
n = number of participants with valid observations;
NC = not calculated;
t_{1/2} = apparent terminal elimination half-life;
tₗₐₛₜ = time of the last quantifiable concentration;
tₘₐₓ = time of the maximum observed concentration;
V_{z}/F = apparent volume of distribution during the terminal phase;
(%)AUCₑₓₜᵣₐₚ = percentage of area under the concentration-time curve due to extrapolation from the last quantifiable concentration to infinity
Geometric mean (CV) [n] statistics presented;
for tₘₐₓ, and tₗₐₛₜ, median (min-max) [n] statistics presented.

Following administration of a single oral dose of 200 mg belumosudil, the metabolite KD025m1 was steadily produced with median tₘₐₓ values of 3.00, 2.46, 3.95, and 4.52 hours for participants with normal hepatic function, mild hepatic impairment, moderate hepatic impairment, and severe hepatic impairment, respectively.

The geometric mean t_{1/2} of KD025m1 increased with increasing severity of hepatic impairment, with values of 2.29, 2.46, 3.06, and 5.36 hours in participants with normal hepatic function, and with mild, moderate, and severe hepatic impairment, respectively.

The AUC_{0-∞} was not calculable for KD025m1 in the normal hepatic function group due to limited quantifiable data. For hepatic impairment groups, geometric mean AUC_{0-∞} values increased from mild, moderate, to severe impairment. The AUC₀₋ₜ and Cₘₐₓ in participants with hepatic impairment were higher than in participants with normal hepatic function, and increased with increasing severity of hepatic impairment. Statistical analysis showed that the ratios of GLSMs (90% CI) for AUC₀₋ₜ and Cₘₐₓ were 3.15 (1.46, 6.79) and 1.63 (0.980, 2.70), respectively, in participants with mild hepatic impairment, 3.56 (1.53, 8.31) and 2.40 (1.36, 4.25), respectively, in participants with moderate hepatic impairment, and 18.6 (5.89, 58.4) and 8.59 (4.72, 15.6) in participants with severe hepatic impairment. These results indicated that hepatic impairment significantly increased KD025m1 exposure, especially in participants with severe hepatic impairment.

As assessed from the within-matched participants CV %, the variability was high for all comparisons for AUC₀₋ₜ and Cₘₐₓ, with values ranging from 37.2% (geometric CV %) to 96.1% (total CV % as participant matching could not be fitted in the model).

The geometric mean MRAUC₀₋ₜ for KD025m1 was higher in participants with hepatic impairment compared to participants with normal hepatic function; however, the associated variability (based on geometric CV %) was also much higher in the mild and moderate hepatic impairment groups, although similar in the severe hepatic impairment group. The geometric mean MRAuc₀₋ₜ (CV %) was 0.00696 (24.1%), 0.0147 (70.3%), 0.0182 (91.6%), and 0.0377 (26.1%) for participants with normal hepatic function, mild hepatic impairment, moderate hepatic impairment, and severe hepatic impairment, respectively.

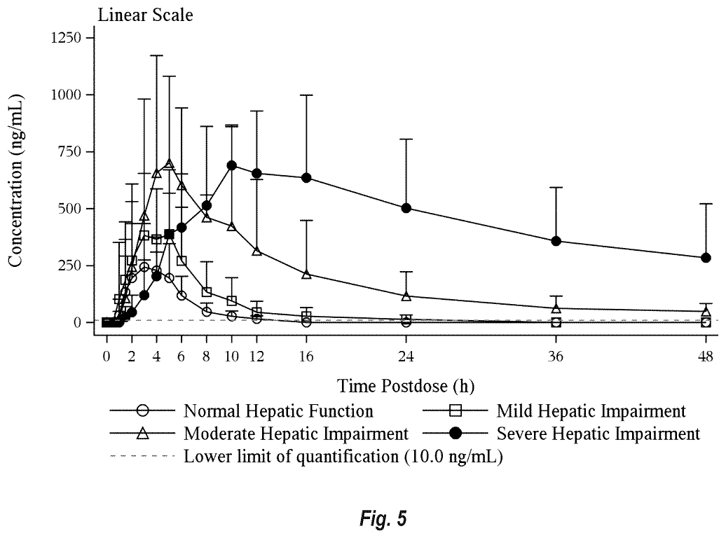

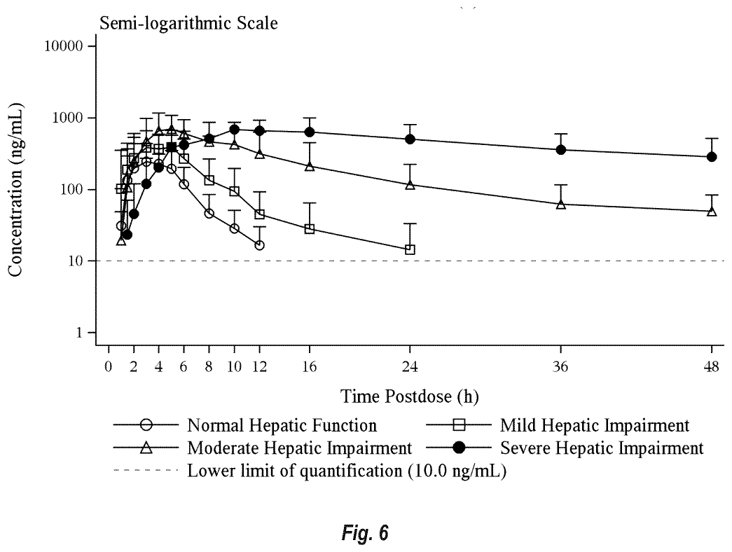

Arithmetic mean (+SD) plasma concentration-time profiles of KD025m2 are presented in FIGS. 5 and 6. The summary and statistical analysis of plasma PK parameters of KD025m2 are presented in Table 3.

TABLE 3
Summary of the Plasma Pharmacokinetic Parameters of KD025 m2

| | Normal Hepatic | Mild Hepatic | Moderate Hepatic | Severe Hepatic |
|---|---|---|---|---|
| | Function | Impairment | Impairment | Impairment |
| Parameter | (N = 14) | (N = 8) | (N = 8) | (N = 6) |
| AUC(0-∞) | 1220 (66.8) [14] | 2040 (120.4) [8] | 7500 (117.5) [7] | NC (NC) [2] |
| (h*ng/mL) | | | | |
| AUC(0-24) | 1160 (61.7) [14] | 1960 (114.0) [8] | 5720 (102.2) [7] | 11000 (38.3) [4] |
| (h*ng/mL) | | | | |
| AUC0-t | 1130 (64.8) [14] | 1940 (123.3) [8] | 6680 (121.9) [7] | 18300 (62.9) [4] |
| (h*ng/mL) | | | | |
| Cmax | 335 (61.6) [14] | 437 (73.1) [8] | 660 (111.1) [7] | 838 (18.8) [4] |
| (ng/mL) | | | | |
| tmax (h) | 3.00 | 2.98 | 5.00 | 11.0 |
| | (1.50-6.00) [14] | (1.92-5.00) [8] | (4.00-10.0) [7] | (6.05-16.0) [4] |
| tlast (h) | 14.0 | 23.9 | 48.0 | 48.0 |
| | (5.97-24.0) [14] | (5.88-48.0) [8] | (16.1-48.0) [7] | (48.0-48.0) [4] |
| t1/2 (h) | 4.06 (114.0) [14] | 4.47 (79.5) [8] | 11.3 (57.4) [7] | 17.7 (37.2) [3] |
| MRAUC | 0.103 (68.8) [14] | 0.135 (73.8) [8] | 0.318 (70.5) [7] | 0.680 (63.5) [4] |
| tlag (h) | NC (NC) [14] | NC (NC) [8] | NC (NC) [7] | NC (NC) [4] |

Matrix: Plasma;
Analyte: KD025 m2
AUC0-∞ = area under the concentration-time curve from time 0 to infinity;
AUC0-t = area under the concentration-time curve from time 0 to the time of the last quantifiable concentration;
CL/F = apparent total clearance;
Cmax = maximum observed concentration;
CV = coefficient of variation (%);
n = number of participants with valid observations;
NC = not calculated;
t1/2 = apparent terminal elimination half-life;
tlast = time of the last quantifiable concentration;
tmax = time of the maximum observed concentration;
V_{z}/F = apparent volume of distribution during the terminal phase;
(%)AUCₑₓₜᵣₐₚ = percentage of area under the concentration-time curve due to extrapolation from the last quantifiable concentration to infinity
Geometric mean (CV) [n] statistics presented;
for tmax, and tlast, median (min-max) [n] statistics presented.

Following administration of a single oral dose of 200 mg belumosudil, the metabolite KD025m2 was steadily produced, with median tₘₐₓ values of 3.00, 2.98, 5.00, and 11.00 hours for participants with normal hepatic function, mild hepatic impairment, moderate hepatic impairment, and severe hepatic impairment, respectively.

The geometric mean t_{1/2} of KD025m2 was similar between participants with normal hepatic function and participants with mild hepatic impairment, with values of 4.06 and 4.47 hours, respectively. The geometric mean t_{1/2} was much longer for participants with moderate and severe hepatic impairment, with values of 11.3 and 17.7 hours, respectively.

Geometric mean PK exposure of KD025m2 was higher in participants with hepatic impairment compared to participants with normal hepatic function. Statistical analysis showed that the effect of mild impairment on the AUC_{0-∞}, AUC₀₋ₜ, and Cₘₐₓ of KD025m2 was not apparent, with the respective ratio of GLSMs (90% CI) of 1.68 (0.748, 3.79), 1.70 (0.793, 3.65), and 1.15 (0.676, 1.96), respectively, and 90% CIs spanning the unity of 1.

However, moderate and severe hepatic impairment significantly increased the PK exposure of KD025m2; the ratios of GLSMs (90% CI) for AUC_{0-∞}, AUC₀₋ₜ, and Cₘₐₓ were 5.06 (2.73, 9.35), 4.95 (2.68, 9.15), and 1.70 (1.00, 2.88), respectively, in participants with moderate hepatic impairment, and for AUC_{0-∞} and Cₘₐₓ were 15.3 (7.34, 32.0) and 2.55 (1.51, 4.31), respectively, in participants with severe hepatic impairment (AUC_{0-∞} was not calculated for the severe group).

As assessed from the within-matched participants CV %, the variability was high for all comparisons for AUC_{0-∞}, AUC₀₋ₜ, and Cₘₐₓ, with values ranging from 32.2% (geometric CV %) to 105.9% (total CV % as participant matching could not be fitted in the model).

The geometric mean MRAUC for KD025m2 was generally similar between normal hepatic function and mild hepatic impairment groups, with values (CV %) of 0.103 (68.8%) and 0.135 (73.8%), respectively. The geometric mean MRAUC was the highest in participants with severe hepatic impairment, followed by participants with moderate hepatic impairment, with values (CV %) of 0.680 (63.5%) and 0.318 (70.5%), respectively.

### Safety Results

There were no reports of deaths, no reports of AEs related to study drug, and no participants discontinued due to an AE during the study.

Overall, 1 participant (12.5%) in the moderate hepatic impairment group reported a total of 2 TEAEs; 1 of which was classified as an SAE. Additionally, 2 participants reported AEs of urinary tract infection on Day −1, which were not considered treatment-emergent as they were prior to dose administration.

There were no clinically significant changes in clinical chemistry, hematology, coagulation, or urinalysis throughout the duration of the study.

During all parts of the study, there were no clinically significant changes in the vital signs parameters for individual participants. Although transient changes in blood pressure and pulse rate were noted at isolated timepoints for some participants, none of these findings were clinically important.

There were no clinically significant changes in the 12-lead ECG parameters for individual participants.

There were no clinically significant findings in the physical examinations performed throughout the study.

### Example 2: United States REZUROCK™ (Belumosudil) FDA Label

### Indications and Usage

REZUROCK is a kinase inhibitor indicated for the treatment of adult and pediatric patients 12 years and older with chronic graft-versus-host disease (chronic GVHD) after failure of at least two prior lines of systemic therapy. (1)

### Dosage and Administration

Recommended Dosage: 200 mg taken orally once daily with food. (2.1)

### Dosage Forms and Strengths

Tablet: 200 mg. (3)

### Contraindications

None. (4)

### Warnings and Precautions

Embryo-Fetal Toxicity: Can cause fetal harm. Advise females of reproductive potential of the potential risk to a fetus and to use effective contraception. (5.1, 8.1, 8.3)

### Adverse Reactions

The most common (≥20%) adverse reactions, including laboratory abnormalities, are infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension. (6.1)

### Drug Interactions

Strong CYP3A Inducers: Increase REZUROCK dosage to 200 mg twice daily. (7.1, 2.3)

Proton Pump Inhibitors: Increase REZUROCK dosage to 200 mg twice daily. (7.1, 2.3)

### Use in Specific Populations

Lactation: Advise not to breastfeed. (8.2)

Moderate or Severe Hepatic Impairment: Avoid use of REZUROCK in patients with moderate or severe hepatic impairment. (2.4, 8.7)

See 17 for PATIENT COUNSELING INFORMATION and FDA-approved patient labeling.

### Full Prescribing Information

### 1 Indications and Usage

REZUROCK is indicated for the treatment of adult and pediatric patients 12 years and older with chronic graft-versus-host disease (chronic GVHD) after failure of at least two prior lines of systemic therapy.

### 2 Dosage and Administration

### 2.1 Recommended Dosage

The recommended dose of REZUROCK is 200 mg given orally once daily until progression of chronic GVHD that requires new systemic therapy.

Instruct the patient on the following:
Swallow REZUROCK tablets whole. Do not cut, crush, or chew tablets.
Take REZUROCK with a meal at approximately the same time each day [see Clinical Pharmacology (12.3)].
If a dose of REZUROCK is missed, instruct the patient to not take extra doses to make up the missed dose.

Treatment with REZUROCK has not been studied in patients with pre-existing severe renal impairment. For patients with pre-existing severe renal or hepatic impairment, consider the risks and potential benefits before initiating treatment with REZUROCK [see Clinical Pharmacology (12.3)].

### 2.2 Dosage Modifications for Adverse Reactions

Monitor total bilirubin, aspartate aminotransferase (AST), and alanine aminotransferase (ALT) at least monthly. Modify the REZUROCK dosage for adverse reactions as per Table 4.

TABLE 4
Recommended Dosage Modifications for REZUROCK for Adverse Reactions

| | | REZUROCK Dosage |
|---|---|---|
| Adverse Reaction | Severity* | Modifications |
| Hepatotoxicity | Grade 3 AST or ALT (5× to | Hold REZUROCK until recovery of |
| [see Adverse | 20 × ULN) or | bilirubin, AST and ALT to Grade |
| Reactions (6.1)] | Grade 2 bilirubin (1.5× to | 0-1, then resume REZUROCK at the |
| | 3 × ULN) | recommended dose. |
| | Grade 4 AST or ALT (more | Discontinue |
| | than 20 × ULN) or | REZUROCK |
| | Grade ≥ 3 bilirubin (more | permanently. |
| | than 3 × ULN) | |
| Other adverse | Grade 3 | Hold REZUROCK until recovery |
| reactions [see | | to Grade 0-1, then resume |
| Adverse | | REZUROCK at the recommended |
| Reactions (6.1)] | | dose level. |
| | Grade 4 | Discontinue |
| | | REZUROCK |
| | | permanently. |

*Based on CTCAE v 4.03

### 2.3 Dosage Modification Due to Drug Interactions

### Strong CYP3A Inducers

Increase the dosage of REZUROCK to 200 mg twice daily when coadministered with strong CYP3A inducers [see Drug Interactions (7.1)].

### Proton Pump Inhibitors

Increase the dosage of REZUROCK to 200 mg twice daily when coadministered with proton pump inhibitors [see Drug Interactions (7.1)].

2.4 Recommended Dosage in Patients with Hepatic Impairment

Avoid use in patients with moderate hepatic impairment (Child-Pugh B) or severe hepatic impairment (Child-Pugh C) without liver GVHD [see Use in Specific Populations (8.7), Clinical Pharmacology (12.3)].

No dosage adjustment is recommended when administering REZUROCK to patients with mild hepatic impairment [see Use in Specific Populations (8.7), Clinical Pharmacology (12.3)].

### 3 Dosage Forms and Strengths

Each 200 mg belumosudil tablet is a pale yellow film-coated oblong tablet debossed with “KDM” on one side and “200” on the other side.

### 4 Contraindications

None.

### 5 Warnings and Precautions

### 5.1 Embryo-Fetal Toxicity

Based on findings in animals and its mechanism of action, REZUROCK can cause fetal harm when administered to a pregnant woman. In animal reproduction studies, administration of belumosudil to pregnant rats and rabbits during the period organogenesis caused adverse developmental outcomes including embryo-fetal mortality and malformations at maternal exposures (AUC) less than those in patients at the recommended dose. Advise pregnant women of the potential risk to a fetus. Advise females of reproductive potential and males with female partners of reproductive potential to use effective contraception during treatment with REZUROCK and for one week after the last dose [see Use in Specific Populations (8.1, 8.3), Nonclinical Toxicology (13.1)].

### 6 Adverse Reactions

### 6.1 Clinical Trial Experience

Because clinical trials are conducted under widely variable conditions, adverse reaction rates observed in clinical trials of a drug cannot be directly compared with rates of clinical trials of another drug and may not reflect the rates observed in practice.

### Chronic Graft Versus Host Disease

In two clinical trials (Study KD025-213 and Study KD025-208), 83 adult patients with chronic GVHD were treated with REZUROCK 200 mg once daily [see Clinical Studies (14.1)]. The median duration of treatment was 9.2 months (range 0.5 to 44.7 months).

Fatal adverse reaction was reported in one patient with severe nausea, vomiting, diarrhea and multi-organ failure.

Permanent discontinuation of REZUROCK due to adverse reactions occurred in 18% of patients. The adverse reactions which resulted in permanent discontinuation of REZUROCK in >3% of patients included nausea (4%). Adverse reactions leading to dose interruption occurred in 29% of patients. The adverse reactions leading to dose interruption in ≥2% were infections (110%), diarrhea (4%), and asthenia, dyspnea, hemorrhage, hypotension, liver function test abnormal, nausea, pyrexia, edema, and renal failure with (2% each).

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension.

Table 5 summarizes the nonlaboratory adverse reactions.

TABLE 5
Nonlaboratory Adverse Reactions in ≥10% Patients
with Chronic GVHD Treated with REZUROCK

REZUROCK
200 mg once daily (N = 83)

| | | |
|---|---|---|
| | All Grades | Grades 3-4 |
| Adverse Reaction | (%) | (%) |

Infections and infestations

| | | |
|---|---|---|
| Infection (pathogen not specified)^{a} | 53 | 16 |
| Viral infection^{†} | 19 | 4 |
| Bacterial infection^{‡} | 16 | 4 |

General disorders and administration site conditions

| | | |
|---|---|---|
| Asthenia^{§} | 46 | 4 |
| Edema^{¶} | 27 | 1 |
| Pyrexia | 18 | 1 |

Gastrointestinal

| | | |
|---|---|---|
| Nausea^{#} | 42 | 4 |
| Diarrhea | 35 | 5 |
| Abdominal pain [symbol] | 22 | 1 |
| Dysphagia | 16 | 0 |

Respiratory, thoracic and mediastinal

| | | |
|---|---|---|
| Dyspnea^{β} | 33 | 5 |
| Cough^{à} | 30 | 0 |
| Nasal congestion | 12 | 0 |

Vascular

| | | |
|---|---|---|
| Hemorrhage^{è} | 23 | 5 |
| Hypertension | 21 | 7 |

Musculoskeletal and connective tissue

| | | |
|---|---|---|
| Musculoskeletal pain^{ð} | 22 | 4 |
| Muscle spasm | 17 | 0 |
| Arthralgia | 15 | 2 |

Nervous system

| | | |
|---|---|---|
| Headache^{ø} | 21 | 0 |

Metabolism and nutrition

| | | |
|---|---|---|
| Decreased appetite | 17 | 1 |

Skin and subcutaneous

| | | |
|---|---|---|
| Rash^{ý} | 12 | 0 |
| Pruritus^{£} | 11 | 0 |

* infection with an unspecified pathogen includes acute sinusitis, device related infection, ear infection, folliculitis, gastroenteritis, gastrointestinal infection, hordeolum, infectious colitis, lung infection, skin infection, tooth infection, urinary tract infection, wound infection, upper respiratory tract infection, pneumonia, conjunctivitis, sinusitis, respiratory tract infection, bronchitis, sepsis, septic shock.
^{†}includes influenza, rhinovirus infection, gastroenteritis viral, viral upper respiratory tract infection, bronchitis viral, Epstein-Barr viremia, Epstein-Barr virus infection, parainfluenzae virus infection, Varicella zoster virus infection, viral infection.
^{‡}includes cellulitis, Helicobacter infection, Staphylococcal bacteremia, catheter site cellulitis, Clostridium difficile colitis, Escherichia urinary tract infection, gastroenteritis Escherichia coli, Pseudomonas infection, urinary tract infection bacterial.
^{§}includes fatigue, asthenia, malaise.
^{¶}includes edema peripheral, generalized edema, face edema, localized edema, edema.
^{#}includes nausea, vomiting. [symbol]
includes abdominal pain, abdominal pain upper, abdominal pain lower.
^{β}includes dyspnea, dyspnea exertional, apnea, orthopnea, sleep apnea syndrome.
^{à}includes cough, productive cough.
^{è}includes contusion, hematoma, epistaxis, increased tendency to bruise, conjunctival hemorrhage, hematochezia, mouth hemorrhage, catheter site hemorrhage, hematuria, hemothorax, purpura.
^{ð}includes pain in extremity, back pain, flank pain, limb discomfort, musculoskeletal chest pain, neck pain, musculoskeletal pain.
^{ø}includes headache, migraine.
^{ý}includes rash, rash maculo-papular, rash erythematous, rash generalized, dermatitis exfoliative.
^{£}includes pruritus, pruritus generalized.

Table 6 summarizes the laboratory abnormalities in REZUROCK.

TABLE 6
Selected Laboratory Abnormalities in Patients
with Chronic GVHD Treated with REZUROCK

| | REZUROCK | | |
|---|---|---|---|
| | 200 mg once daily | | |
| | | Grade | Grade |
| | Grade | 2-4 | 3-4 |
| | 0-1 | Max | Max |
| | Baseline | Post | Post |
| Parameter | (N) | (%) | (%) |
| Chemistry | | | |
| Phosphate decreased | 76 | 28 | 7 |
| Gamma Glutamyl Transferase increased | 47 | 21 | 11 |
| Calcium decreased | 82 | 12 | 1 |
| Alkaline Phosphatase increased | 80 | 9 | 0 |
| Potassium increased | 82 | 7 | 1 |
| Alanine Aminotransferase increased | 83 | 7 | 2 |
| Creatinine increased | 83 | 4 | 0 |
| Hematology | | | |
| Lymphocytes decreased | 62 | 29 | 13 |
| Hemoglobin decreased | 79 | 11 | 1 |
| Platelets decreased | 82 | 10 | 5 |
| Neutrophil Count decreased | 83 | 8 | 4 |

### 7 Drug Interactions

### 7.1 Effect of Other Drugs on REZUROCK

### Strong CYP3A Inducers

Coadministration of REZUROCK with strong CYP3A inducers decreases belumosudil exposure [see Clinical Pharmacology (12.3)], which may reduce the efficacy of REZUROCK. Increase the dosage of REZUROCK when coadministered with strong CYP3A inducers [see Dosage and Administration (2.3)].

### Proton Pump Inhibitors

Coadministration of REZUROCK with proton pump inhibitors decreases belumosudil exposure [see Clinical Pharmacology (12.3)], which may reduce the efficacy of REZUROCK. Increase the dosage of REZUROCK when coadministered with proton pump inhibitors [see Dosage and Administration (2.3)].

### 8 Use in Specific Populations

### 8.1 Pregnancy

### Risk Summary

Based on findings from animal studies and the mechanism of action [see Clinical Pharmacology (12.1)], REZUROCK can cause fetal harm when administered to pregnant women. There are no available human data on REZUROCK use in pregnant women to evaluate for a drug-associated risk. In animal reproduction studies, administration of belumosudil to pregnant rats and rabbits during the period of organogenesis resulted in adverse developmental outcomes, including alterations to growth, embryo-fetal mortality, and embryo-fetal malformations at maternal exposures (AUC) approximately ≥3- (rat) and ≥0.07 (rabbit) times the human exposure (AUC) at the recommended dose (see Data). Advise pregnant women and females of reproductive potential of the potential risk to the fetus.

In the U.S. general population, the estimated background risk of major birth defects and miscarriage in clinically recognized pregnancies is 2 to 4% and 15 to 20%, respectively.

### Data

### Animal Data

Embryo-fetal development studies were conducted in rats with administration of belumosudil to pregnant animals during the period of organogenesis at oral doses of 25, 50, 150, and 300 mg/kg/day in a pilot study and doses of 15, 50, and 150 mg/kg/day in a pivotal study. In the pilot study, maternal toxicity and embryo-fetal developmental effects were observed. Maternal toxicity (reduced body weight gain) occurred at 150 and 300 mg/kg/day doses. Increased post-implantation loss occurred at 50 and 300 mg/kg/day. Fetal-malformations were observed at ≥50 mg/kg/day and included absence of anus and tail, omphalocele, and dome shaped head. The exposure (AUC) at 50 mg/kg/day in rats is approximately 3 times the human exposure at the recommended dose of 200 mg.

In an embryo-fetal developmental study in rabbits, pregnant animals administered oral doses of belumosudil at 50, 125, and 225 mg/kg/day during the period of organogenesis resulted in maternal toxicity and embryo-fetal developmental effects. Maternal toxicity (body weight loss and mortality) was observed at doses ≥125 mg/kg/day. Embryo-fetal effects were observed at doses ≥50 mg/kg/day and included spontaneous abortion, increased post-implantation loss, decreased percentage of live fetuses, malformations, and decreased fetal body weight. Malformations included those in the tail (short), ribs (branched, fused or deformed), stemebrae (fused), and neural arches (fused, misaligned, and deformed). The exposure (AUC) at 50 mg/kg/day in rabbits is approximately 0.07 times the human exposure at the recommended dose of 200 mg.

### 8.2 Lactation

### Risk Summary

There are no data available on the presence of belumosudil or its metabolites in human milk or the effects on the breastfed child, or milk production. Because of the potential for serious adverse reactions from belumosudil in the breastfed child, advise lactating women not to breastfeed during treatment with REZUROCK and for one week after the last dose.

### 8.3 Females and Males of Reproductive Potential

REZUROCK can cause fetal harm when administered to a pregnant woman [see Use in Specific Populations (8.1)].

### Pregnancy Testing

Verify the pregnancy status of females of reproductive potential prior to initiating treatment with REZUROCK. Contraception

### Females

Advise females of reproductive potential to use effective contraception during treatment with REZUROCK and for one week after the last dose of REZUROCK. If this drug is used during pregnancy or if the patient becomes pregnant while taking this drug, the patient should be informed of the potential hazard to a fetus.

### Males

Advise males with female partners of reproductive potential to use effective contraception during treatment with REZUROCK and for one week after the last dose of REZUROCK.

### Infertility

### Females

Based on findings from rats, REZUROCK may impair female fertility. The effect on fertility is reversible [see Nonclinical Toxicology (13.1)].

### Males

Based on findings from rats and dogs, REZUROCK may impair male fertility. The effects on fertility are reversible [see Nonclinical Toxicology (13.1)].

### 8.4 Pediatric Use

The safety and effectiveness of REZUROCK have been established in pediatric patients 12 years and older. Use of REZUROCK in this age group is supported by evidence from adequate and well-controlled studies of REZUROCK in adults with additional population pharmacokinetic data demonstrating that age and body weight had no clinically meaningful effect on the pharmacokinetics of drug substance, that the exposure of drug substance is expected to be similar between adults and pediatric patients age 12 years and older, and that the course of disease is sufficiently similar in adult and pediatric patients to allow extrapolation of data in adults to pediatric patients.

The safety and effectiveness of REZUROCK in pediatric patients less than 12 years old have not been established.

### 8.5 Geriatric Use

Of the 186 patients with chronic GVHD in clinical studies of REZUROCK, 26% were 65 years and older. No clinically meaningful differences in safety or effectiveness of REZUROCK were observed in comparison to younger patients.

### 8.6 Renal Impairment

Treatment with REZUROCK has not been studied in patients with pre-existing severe renal impairment. For patients with pre-existing severe renal impairment, consider the risks and potential benefits before initiating treatment with REZUROCK [see Dosage and Administration (2.1) and Clinical Pharmacology (12.3)].

### 8.7 Hepatic Impairment

Avoid use in patients with moderate hepatic impairment (Child-Pugh B) or severe hepatic impairment (Child-Pugh C) without liver GVHD [see Dosage and Administration (2.4), Clinical Pharmacology (12.3)].

No dosage adjustment is recommended for patients with mild hepatic impairment (Child-Pugh A) [see Dosage and Administration (2.4), Clinical Pharmacology (12.3)].

### 11 Description

Belumosudil is a kinase inhibitor. The active pharmaceutical ingredient is belumosudil mesylate with the molecular formula C₂₇H₂₈N₆O₅S and the molecular weight is 548.62 g/mol. The chemical name for belumosudil mesylate is 2-{3-[4-(1H-indazol-5-ylamino)-2-quinazolinyl]phenoxy}-N-(propan-2-yl) acetamide methanesulfonate (1:1). The chemical structure is as follows:

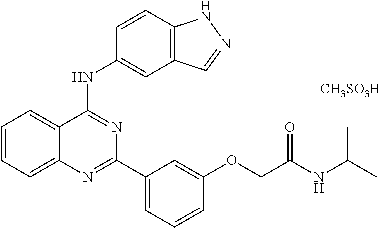 [SMILES: CC(C)NC(=O)COc1cccc(-c2nc(Nc3ccc4[nH]ncc4c3)c3ccccc3n2)c1 ; "CH3SO3H"]

Belumosudil mesylate is a yellow powder that is practically insoluble in water, slightly soluble in methanol and DMF and soluble in DMSO.

REZUROCK tablets are for oral administration. Each tablet contains 200 mg of the free base equivalent to 242.5 mg of belumosudil mesylate. The tablet also contains the following inactive ingredients: microcrystalline cellulose, hypromellose, croscarmellose sodium, colloidal silicon dioxide, and magnesium stearate.

The tablet film consists of polyvinyl alcohol, polyethylene glycol, talc, titanium dioxide and yellow iron oxide.

### 12 Clinical Pharmacology

### 12.1 Mechanism of Action

Belumosudil is an inhibitor of rho-associated, coiled-coil containing protein kinase (ROCK) which inhibits ROCK2 and ROCK1 with IC₅₀ values of approximately 100 nM and 3 μM, respectively. Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. In vivo, belumosudil demonstrated activity in animal models of chronic GVHD.

### 12.2 Pharmacodynamics

Belumosudil exposure-response relationships and the time course of pharmacodynamic response are not established.

### Cardiac Electrophysiology

At 2.4 times the maximum exposure for approved recommended dose, REZUROCK does not prolong the QT interval to any clinically relevant extent.

### 12.3 Pharmacokinetics

The following pharmacokinetic parameters are presented for chronic GVHD patients administered belumosudil 200 mg once daily, unless otherwise specified. The mean (% coefficient of variation, % CV) steady-state AUC and Cₘₐₓ of belumosudil was 22,700 (48%) h·ng/mL and 2390 (44%) ng/mL, respectively. Belumosudil Cₘₐₓ and AUC increased in an approximately proportional manner over a dosage range of 200 and 400 mg (1 to 2 times once daily recommended dosage). The accumulation ratio of belumosudil was 1.4.

### Absorption

Median Tₘₐₓ of belumosudil at steady state was 1.26 to 2.53 hours following administration of 200 mg once daily or twice daily in patients. The mean (% CV) bioavailability was 64% (17%) following a single belumosudil dose in healthy subjects.

### Effect of Food

Belumosudil Cₘₐₓ and AUC increased 2.2 times and 2 times, respectively, following administration of a single belumosudil dose with a high-fat and high-calorie meal (800 to 1,000 calories with approximately 50% of total caloric content of the meal from fat) compared to the fasted state in healthy subjects. Median Tₘₐₓ was delayed 0.5 hours.

### Distribution

The geometric mean volume of distribution after a single dose of belumosudil in healthy subjects was 184 L (geo CV % 67.7%).

Belumosudil binding to human serum albumin and human α₁-acid glycoprotein was 99.9% and 98.6%, respectively, in vitro.

### Elimination

The mean (% CV) elimination half-life of belumosudil was 19 hours (39%), and clearance was 9.83 L/hours (46%) in patients.

### Metabolism

Belumosudil is primarily metabolized by CYP3A4 and to a lesser extent by CYP2C8, CYP2D6, and UGT1A9, in vitro.

### Excretion

Following a single oral dose of radiolabeled belumosudil in healthy subjects, 85% of radioactivity was recovered in feces (30% as unchanged) and less than 5% in urine.

### Specific Populations

No clinically significant differences in belumosudil pharmacokinetics were observed with regard to age (18 to 77 years), sex, weight (38.6 to 143 kg), or mild to moderate renal impairment (eGFR ≥60 and <90 mL/min/1.72 m² to eGFR ≥30 and <60 mL/min/1.72 m²). The effect of severe renal impairment on the pharmacokinetics of belumosudil has not been studied.

Patients with Hepatic Impairment

Following a single 200 mg dose of belumosudil, changes in belumosudil exposure in subjects with varying degrees of hepatic impairment based on Child-Pugh score without liver GVHD relative to subjects with normal hepatic function is shown in Table 7.

TABLE 7
Effect of Varying Degrees of Hepatic
Impairment on Belumosudil Exposure

| | Changes in Belumosudil Exposure in | | | |
|---|---|---|---|---|
| | Subjects with Hepatic Impairment Compared | | | |
| | to Subjects with Normal Hepatic Function | | | |
| | Total (Free + Bound) | | | |
| Hepatic Impairment | Concentrations | | Free Concentrations | |
| Category | Cₘₐₓ | AUC | Cₘₐₓ | AUC |
| Mild (Child-Pugh A) | 1.2-fold | 1.4-fold | 14% | 19% |
| | increase | increase | decrease | decrease |
| Moderate (Child-Pugh B) | 6% | 1.5-fold | 12% | 1.4-fold |
| | decrease | increase | decrease | increase |
| Severe (Child-Pugh C) | 1.3-fold | 4.2-fold | 5.4-fold | 16-fold |
| | increase | increase | increase | increase |

### Drug Interaction Studies

### Clinical Studies and Model-Informed Approaches

### Effects of Other Drugs on Belumosudil

Strong Cytochrome P450 (CYP) 3A Inhibitors: There was no clinically meaningful effect on belumosudil exposure when coadministered with itraconazole in healthy subjects.

Strong CYP3A Inducers: Coadministration of rifampin decreased belumosudil Cₘₐₓ by 59% and AUC by 72% in healthy subjects.

Moderate CYP3A Inducers: Coadministration of efavirenz is predicted to decrease belumosudil Cₘₐₓ by 19% and AUC by 35% in healthy subjects.

Proton Pump Inhibitors: Coadministration of rabeprazole decreased belumosudil Cₘₐₓ by 87% and AUC by 80%, and omeprazole decreased belumosudil Cₘₐₓ by 68% and AUC by 47% in healthy subjects.

### Effects of Belumosudil on Other Drugs

CYP3A Substrates: Coadministration of belumosudil is predicted to increase midazolam (a sensitive CYP3A substrate) Cₘₐₓ and AUC approximately 1.3- and 1.5-fold, respectively.

CYP2C9 Substrates: Coadministration of belumosudil is not expected to have clinically meaningful effect on the exposure of CYP2C9 substrates (such as warfarin).

CYP2C8 Substrates: Coadministration of belumosudil is not expected to have clinically meaningful effect on the exposure of CYP2C8 substrates that are not an OATP1B1 substrate.

### In Vitro Studies

Transporter Systems: Belumosudil is a substrate of P-gp. Belumosudil inhibits BCRP, P-gp, and OATP1B1 at clinically relevant concentrations.
Enzymes Systems: Belumosudil is an inhibitor of CYP1A2, CYP2C19, CYP2D6, UGT1A1 and UGT1A9.

### 13 Nonclinical Toxicology

### 13.1 Carcinogenesis, Mutagenesis, Impairment of Fertility

### Carcinogenesis

Carcinogenicity studies have not been conducted with belumosudil.

### Mutagenesis

Belumosudil was not genotoxic in an in vitro bacterial mutagenicity (Ames) assay, in vitro chromosome aberration assay in human peripheral blood lymphocytes (HPBL) or an in vivo rat bone marrow micronucleus assay.

### Impairment of Fertility

In a combined male and female rat fertility study, belumosudil-treated male animals were mated with untreated females, or untreated males were mated with belumosudil-treated females. Belumosudil was administered orally at doses of 50, 150 or 275 mg/kg/day to male rats 70 days prior to and throughout the mating period, and to female rats 14 days prior to mating and up to Gestation Day 7. At the dose of 275 mg/kg/day, adverse findings in female rats (treated with belumosudil or untreated but mated with treated males) included increased pre- or post-implantation loss and decreased number of viable embryos. Administration of belumosudil to male rats at a dose of 275 mg/kg/day resulted in abnormal sperm findings (reduced motility, reduced count, and increased percentage of abnormal sperm), and testes/epididymis organ changes (reduced weight and degeneration).

Fertility was reduced in both treated males or females at the 275 mg/kg/day dose and reached statistical significance in males. Adverse changes in male and female reproductive organs also occurred in general toxicology studies; findings included spermatozoa degeneration at a belumosudil dose of 35 mg/kg/day in dogs and decreased follicular development in ovaries at 275 mg/kg/day in rats. Changes were partially or fully reversed during the recovery period. The exposure (AUC) at the doses of 35 mg/kg/day in dogs, and 275 mg/kg/day in rats is 0.5 times and 8-9 times, respectively, the clinical exposure at the recommended dose of 200 mg daily.

### 14 Clinical Studies

### 14.1 Chronic Graft Versus Host Disease

Study KD025-213 (NCT03640481) was a randomized, open-label, multicenter study of REZUROCK for treatment of patients with chronic GVHD who had received 2 to 5 prior lines of systemic therapy and required additional treatment. Patients were excluded from the studies if platelets were <50×10⁹/L; absolute neutrophil count <1.5×10⁹/L; AST or ALT >3×ULN; total bilirubin >1.5×ULN; QTc(F) >480 ms; eGFR <30 mL/min/1.73 m2; or FEV1≤39%. There were 66 patients treated with REZUROCK 200 mg taken orally once daily. Concomitant treatment with supportive care therapies for chronic GVHD was permitted. Concomitant treatment with GVHD prophylaxis and standard care systemic chronic GVHD therapies was permitted as long as the subject has been on a stable dose for at least 2 weeks prior to study. Initiation of new systemic chronic GVHD therapy while on study was not permitted.

Demographics and baseline characteristics are summarized in Table 8.

TABLE 8
Demographics and Baseline Characteristics
of Patients with Chronic GVHD

| | REZUROCK | |
|---|---|---|
| | 200 mg once | |
| | daily (N = 65) | |
| Age, Median, Years (minimum, maximum) | 53 | (21, 77) |
| Age ≥ 65 Years, n (%) | 17 | (26) |
| Male, n (%) | 42 | (65) |
| Race, n (%) | | |
| White | 54 | (83) |
| Black | 6 | (9) |
| Other or Not Reported | 5 | (8) |
| Median (range) time (months) from | 25.3 | (1.9, 162.4) |
| Chronic GVHD Diagnosis | | |
| ≥4 Organs Involved, n (%) | 31 | (48) |
| Median (range) Number of Prior Lines | 3 | (2, 6) |
| of Therapy | | |
| Number of Prior Lines of Therapy, n (%) | | |
| 2 | 23 | (35) |
| 3 | 12 | (19) |
| 4 | 15 | (23) |
| ≥5 | 15 | (23) |
| Prior chronic GVHD treatment with | 21 | (32) |
| ibrutinib, n (%) | | |
| Prior chronic GVHD treatment with | 20 | (31) |
| ruxolitinib, n (%) | | |
| Refractory to Last Therapy, n (%*) | 43/55 | (78) |
| Severe chronic GVHD, n (%) | 46 | (71) |
| Median (range) Global Severity Rating | 7 | (2, 9) |
| Median (range) Lee Symptom Scale Score | 27 | (7, 56) |
| at baseline | | |
| Median (range) Corticosteroid dose at | 0.19 | (0.03, 0.95) |
| baseline (PE/kg)^{†} | | |

* Denominator excludes patients with unknown status
^{†}Prednisone equivalents/kilogram

The efficacy of REZUROCK was based on overall response rate (ORR) through Cycle 7 Day 1 where overall response included complete response or partial response according to the 2014 NIH Response Criteria. The ORR results are presented in Table 9. The ORR was 75% (95% CI: 63, 85). The median duration of response, calculated from first response to progression, death, or new systemic therapies for chronic GVHD, was 1.9 months (95% CI: 1.2, 2.9). The median time to first response was 1.8 months (95% CI: 1.0, 1.9). In patients who achieved response, no death or new systemic therapy initiation occurred in 62% (95% CI: 46, 74) of patients for at least 12 months since response.

TABLE 9
Overall Response Rate through Cycle 7 Day 1 for
Patients with Chronic GVHD in Study KD025-213

| | REZUROCK |
|---|---|
| | 200 mg once daily |
| | (N = 65) |
| Overall Response Rate (ORR) | 49 (75%) |
| 95% Confidence Interval* | (63%, 85%) |
| Complete Response | 4 (6%) |
| Partial Response | 45 (69%) |

*Estimated using Clopper-Pearson method

ORR results were supported by exploratory analyses of patient-reported symptom bother which showed at least a 7-point decrease in the Lee Symptom Scale summary score through Cycle 7 Day 1 in 52% (95% CI: 40, 65) of patients.

### 16 How Supplied/Storage and Handling

REZUROCK 200 mg tablets are supplied as pale yellow film-coated oblong tablets containing 200 mg of belumosudil (equivalent to 242.5 mg belumosudil mesylate). Each tablet is debossed with “KDM” on one side and “200” on the other side and is packaged as follows:

200 mg tablets in 30 count bottle: NDC 79802-200-30

Store at room temperature, 20° C. to 25° C. (68° F. to 77° F.); excursions permitted from 15° C. to 30° C. (59° F. to 86° F.) [see USP Controlled Room Temperature].

Dispense to patient in original container only. Store in original container to protect from moisture. Replace cap securely each time after opening. Do not discard desiccant.

### 17 Patient Counseling Information

Advise the patient to read the FDA-approved patient labeling (Patient Information).

### Embryo-Fetal Toxicity:

Advise pregnant women and females of reproductive potential of the potential risk to a fetus. Advise females of reproductive potential to inform their healthcare provider of a known or suspected pregnancy [see Warnings and Precautions (5.1), Use in Specific Populations (8.1, 8.3)].
Advise females of reproductive potential to use effective contraceptive during treatment with REZUROCK and for one week after the last dose [see Warnings and Precautions (5.1)].
Advise males with female partners of reproductive potential to use effective contraceptive during treatment with REZUROCK and for one week after the last dose [see Use in Specific Populations (8.3)].

### Lactation

Advise women not to breastfeed during treatment with REZUROCK and for one week after the last dose [see Use in Specific Populations (8.2)].

### Infertility

Advise males and females of reproductive potential that REZUROCK may impair fertility [see Use in Specific Populations (8.3)].

### Administration

Inform patients to take REZUROCK orally once daily with food according to their physician's instructions and that the oral dosage (tablets) should be swallowed whole with a glass of water without cutting, crushing or chewing the tablets approximately the same time each day [see Dosage and Administration (2.1)].
Advise patients that in the event of a missed daily dose of REZUROCK, it should be taken as soon as possible on the same day with a return to the normal schedule the following day. Patients should not take extra doses to make up the missed dose [see Dosage and Administration (2.1)].

### Drug Interactions

Advise patients to inform their health care providers of all concomitant medications, including prescription medicines, over-the-counter drugs, vitamins, and herbal products [see Drug Interactions (7)].

Although the present invention has been described in some detail by way of illustration and example for purposes of clarity and understanding, the descriptions and examples should not be construed as limiting the scope of the invention. The disclosures of all patent and scientific literature cited herein are expressly incorporated herein in their entirety by reference.

What is claimed is:

1. A method of treating chronic graft-versus-host disease (cGVHD) in a patient in need thereof, comprising the steps of:
(a) verifying the patient's hepatic function; and
(b) administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is normal hepatic function or mild hepatic impairment (A on the Child-Pugh scale).

2. The method of claim 1, wherein the patient's hepatic function is normal.

3. The method of claim 1, wherein the patient's hepatic function is mild hepatic impairment.

4. The method of claim 1, further comprising the step of:
(c) ceasing the administration of the Compound to the patient when the patient's hepatic function indicates an increase in hepatic impairment.

5. The method of claim 4, wherein the patient's hepatic function changes from normal hepatic function to moderate or severe hepatic impairment (B or C on the Child-Pugh scale).

6. The method of claim 4, wherein the patient's hepatic function changes from mild hepatic impairment (A on the Child-Pugh scale) to moderate or severe hepatic impairment (B or C on the Child-Pugh scale).

7. The method of any one of claims 1-6, wherein normal hepatic function is defined as the patient scoring 0 to 4 points on the Child-Pugh scale, wherein mild hepatic impairment is defined as the patient scoring 5 or 6 points on the Child-Pugh scale, wherein moderate hepatic impairment is defined as the patient scoring 7 to 9 points on the Child-Pugh scale, and wherein severe hepatic impairment is defined as the patient scoring 10 to 14 points on the Child-Pugh scale.

8. A method of treating chronic graft-versus-host disease (cGVHD) in a patient in need thereof, comprising the steps of:
(a) verifying the patient's Child-Pugh score; and
(b) administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 5 or 6.

9. The method of claim 8, further comprising the step of:
(c) ceasing the administration of the Compound to the patient when the patient's Child-Pugh score increases.

10. The method of claim 8, further comprising the step of:
(c) ceasing the administration of the Compound to the patient when the patient's Child-Pugh score increases to 7 or greater.

11. A method of treating chronic graft-versus-host disease (cGVHD) in a patient in need thereof, comprising the steps of:
(a) verifying the patient's Child-Pugh score; and
(b) administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 0 to 4.

12. The method of claim 11, further comprising the step of:
(c) ceasing the administration of the Compound to the patient when the patient's Child-Pugh score increases to 7 or greater.

13. A method of treating chronic graft-versus-host disease (cGVHD) in a patient in need thereof, comprising the steps of:
(a) verifying the patient's hepatic function; and
(b) administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is not moderate or severe hepatic impairment (B or C on the Child-Pugh scale).

14. A method of treating chronic graft-versus-host disease (cGVHD) in a patient in need thereof, comprising the steps of:
(a) verifying the patient's Child-Pugh score; and
(b) administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is not 7 to 14 (B or C on the Child-Pugh scale).

15. A method of treating chronic graft-versus-host disease (cGVHD) in a patient in need thereof, comprising the steps of:
(a) verifying the patient's hepatic function; and
(b) not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is moderate or severe hepatic impairment (B or C on the Child-Pugh scale).

16. A method of treating chronic graft-versus-host disease (cGVHD) in a patient in need thereof, comprising the steps of:
(a) verifying the patient's Child-Pugh score; and
(b) not administering 2-{3-[4-(1H-indazol-5-ylamino)-2-quinazolinyl]phenoxy}-N-(propan-2-yl) acetamide, or a pharmaceutically acceptable salt thereof (Compound) to the patient when the Child-Pugh score of the patient is 7 to 14 (B or C on the Child-Pugh scale).

17. A method of treating chronic graft-versus-host disease (cGVHD) in a patient in need thereof, comprising determining whether the patient has normal hepatic function or mild hepatic impairment, and when the patient is found to have normal hepatic function or mild hepatic impairment, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

18. The method of claim 17, wherein the administration of Compound to the patient is ceased when the patient's hepatic function indicates an increase in hepatic impairment.

19. The method of claim 17, wherein the patient's hepatic function changes from normal hepatic function or mild hepatic impairment to moderate or severe hepatic impairment.

20. A method of treating chronic graft-versus-host disease (cGVHD) in a patient in need thereof, comprising determining the patient's Child-Pugh score, and when the patient is found to have a Child-Pugh score of 5 or 6, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

21. The method of claim 20, wherein the administration of Compound to the patient is ceased when the patient's Child-Pugh score increases to 7 or greater.

22. A method of treating chronic graft-versus-host disease (cGVHD) in a patient in need thereof, comprising determining whether the patient has moderate or severe hepatic impairment, and when the patient is found not to have moderate or severe hepatic impairment, administering to the patient a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound).

23. A method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient has normal hepatic function or mild hepatic impairment.

24. The method of claim 23, wherein the administration of Compound to the patient is ceased when the patient's hepatic function indicates an increase in hepatic impairment.

25. The method of claim 24, wherein the patient's hepatic function changes from normal hepatic function or mild hepatic impairment to moderate or severe hepatic impairment.

26. A method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient does not have moderate or severe hepatic impairment.

27. A method of treating chronic graft-versus-host disease (cGVHD) in a patient in need thereof, comprising administering to a patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (Compound), wherein the patient has a score of A on the Child Pugh scale.

28. The method of claim 27, wherein the administration of Compound to the patient is ceased when the patient's hepatic function indicates an increase in hepatic impairment.

29. The method of claim 28, wherein the patient's hepatic function changes from a score of A to a score of B or C on the Child-Pugh scale.

30. A method of treating chronic graft-versus-host disease (cGVHD) in a patient in need thereof, comprising administering to a patient in need thereof a therapeutically effective amount of Compound, wherein the patient does not have score of B or C on the Child-Pugh scale.

31. A method of treating chronic graft-versus-host disease (cGVHD) in a patient in need thereof, comprising the steps of:
(a) measuring the patient's hepatic function a first time;
(b) administering 2-{3-[4-(1H-indazol-5-ylamino)-2-quinazolinyl]phenoxy}-N-(propan-2-yl) acetamide, or a pharmaceutically acceptable salt thereof (Compound) to the patient when the hepatic function of the patient is mild hepatic impairment (A on the Child-Pugh scale) and not administering Compound when the hepatic function of the patient is moderate or severe hepatic impairment (B or C on the Child-Pugh scale);
(c) measuring the patient's hepatic function a second time, wherein the second time is at least a month after the first time; and
(d) ceasing administration of the Compound when the hepatic function of the patient is moderate or severe hepatic impairment (B or C on the Child-Pugh scale),
wherein measuring the hepatic function comprises measurement of at least one of the following: bilirubin, aspartate aminotransferase (AST), and alanine aminotransferase (ALT).

32. The method of claim 31, wherein the second time is a month after the first time.

33. The method of claim 31, wherein the patient does not have liver cGVHD.

34. The method of any one of claims 1-32, wherein the cGVHD does not comprise liver cGVHD.

35. The method of any one of claims 1-32, wherein patient is without liver cGVHD.

36. The method of any one of the preceding claims, wherein the patient is monitored for hepatic function at least monthly.

37. The method of any one of the preceding claims, wherein the patient is monitored for bilirubin, aspartate aminotransferase (AST), and alanine aminotransferase (ALT) at least monthly.

38. The method of any one of the preceding claims, wherein the Compound comprises belumosudil mesylate salt.

39. The method of any one of the preceding claims, wherein the patient has failed at least two prior lines of systemic therapy for the cGVHD.

40. The method of any one of the preceding claims, wherein the Compound is administered to the patient at a dose of 200 mg once daily.

41. The method of any one of the preceding claims, wherein the Compound is administered to the patient at a dose of 200 mg twice daily.

42. The method of claim 40 or 41, wherein the Compound is administered daily with food.
